SEQUENCE LISTING (81 sequences)

SEQ ID NO: 1 - DNA, 51 bp, Homo sapiens
  misc_feature (24)..(25): ACVR1C_P363_F  methylation site for activin A receptor, type IC (ACVR1C)
ggtccttaagtccaaccaggttgcgctgtgagagccccgcgggcttcctac

SEQ ID NO: 2 - DNA, 50 bp, Homo sapiens
  misc_feature (22)..(23): AR_P54_R methylation site for homo sapiens androgen receptor
aggaggccggcccggtgggggcgggacccgactcgcaaactgttgcattt

SEQ ID NO: 3 - DNA, 49 bp, Homo sapiens
  misc_feature (25)..(26): ATP10A_P147_F methylation site for Homo sapiens ATPase
ccactttcagattccgttgttgggcgaactagaccgtttcctttccacc

SEQ ID NO: 4 - DNA, 56 bp, Homo sapiens
  misc_feature (31)..(32): BCL2L2_P280_F methyation site for Homo sapiens BCL2-like 2 (BCL2L2)
ctggaaaagttcaacaagtgcatggaacatcggaaacctcctgaaaatgctaaatt

SEQ ID NO: 5 - DNA, 42 bp, Homo sapiens
  misc_feature (23)..(24): BDNF_P259_R methylation site for brain-derived neurotrophic factor (BDNF)
tgtcaggctagggcgggaagaccgctggggaacttgttgctt

SEQ ID NO: 6 - DNA, 58 bp, Homo sapiens
  misc_feature (31)..(32): CALCA_E174_R methylation site for Homo sapiens calcitonin/calcitonin-related polypeptide, alpha (CALCA), transcript variant 2
tccaacctagggcacgagcctggtataaatcgcggactaacagagactatctgatgaa

SEQ ID NO: 7 - DNA, 54 bp, Homo sapiens
  misc_feature (29)..(30): CASP10_E139_F methylation site for  caspase 10, apoptosis-related cysteine peptidase (CASP10)
tttgttttcaggcaatttccctgagaaccgtttacttccagaagattggtggag

SEQ ID NO: 8 - DNA, 57 bp, Homo sapiens
  misc_feature: CASP10_P334_F methylation site for
tgtggacataagaaagggttaacatggccgacaactatttcatgagctttttggctt

SEQ ID NO: 9 - DNA, 59 bp, Homo sapiens
  misc_feature: CCR5_P630_R methylation site for Homo sapiens chemokine (C-C motif) receptor 5 (CCR5)
acttctaaacaccattacattgggattcgaatttcaacatgaatttttggggaacacaa

SEQ ID NO: 10 - DNA, 50 bp, Homo sapiens
  misc_feature (28)..(29): CD34_P780_R  methylation site for Homo sapiens CD34 antigen (CD34), transcript variant 1
ggcagcctagtcttggggacgtagagacgggagaaaggagaagccagcct

SEQ ID NO: 11 - DNA, 51 bp, Homo sapiens
  misc_feature (31)..(32): CD44_P87_F methylaton site for Homo sapiens CD44 antigen (Indian blood group) (CD44), transcript variant 2
cttgctccagccggattcagagaaatttagcgggaaaggagaggccaaagg

SEQ ID NO: 12 - DNA, 53 bp, Homo sapiens
  misc_feature: CDH13_E102_F methylation site for Homo sapiens cadherin 13
gtgcatgaatgaaaacgccgccgggcgcttctagtcggacaaaatgcagccga

SEQ ID NO: 13 - DNA, 51 bp, Homo sapiens
  misc_feature: CDK10_P199_R methylation site for Homo sapiens cyclin-dependent kinase (CDC2-like) 10 (CDK10)
cctggaagaccttcacctgggtaatcgccgtggcctcccactacggcgcag

SEQ ID NO: 14 - DNA, 48 bp, Homo sapiens
  misc_feature: COL4A3_P545_F methylation site for Homo sapiens collagen, type IV, alpha 3 (Goodpasture antigen) (COL4A3)
ggcgccttacctgtggggacgcccgcagcgccaggagctgccgccttg

SEQ ID NO: 15 - DNA, 45 bp, Homo sapiens
  misc_feature (21)..(22): DDR1_E23_R methylation site for Homo sapiens discoidin domain receptor family, member 1 (DDR1)
ttcccctcgtgggccctgagcgggactgcagccagccccctgggg

SEQ ID NO: 16 - DNA, 53 bp, Homo sapiens
  misc_feature (26)..(27): DKFZP564O0823_P386_F methylation site for Homo sapiens DKFZP564O0823 protein (DKFZP564O0823)
gtggatgagggtttaatgatgtacacgcagaagtgttttgacaaatgaagaag

SEQ ID NO: 17 - DNA, 57 bp, Homo sapiens
  misc_feature: DLC1_E276_F methylation site for Homo sapiens deleted in liver cancer 1 (DLC1)
agtccatagcgtcttacctagacaacgaggagctgaaacgccaaggcatgacactgc

SEQ ID NO: 18 - DNA, 53 bp, Homo sapiens
  misc_feature: EMR3_E61_F methylation site for Homo sapiens egf-like module containing, mucin-like, hormone receptor-like 3 (EMR3)
agcaaactgcttcccctctttcgccatcagactcatggttctgcttttcgttt

SEQ ID NO: 19 - DNA, 48 bp, Homo sapiens
  misc_feature: ERG_E28_F methylation site for Homo sapiens v-ets erythroblastosis virus E26 oncogene like (avian) (ERG)
aaaatccagcttacctgagcgccgctcctcttctctcatgtccctcgg

SEQ ID NO: 20 - DNA, 48 bp, Homo sapiens
  misc_feature: FRZB_E186_R methylation site for Homo sapiens frizzled-related protein (FRZB)
caggatggggcagggtgcagccgcgcagtggacgccaaaaggcccgct

SEQ ID NO: 21 - DNA, 51 bp, Homo sapiens
  misc_feature (20)..(21): GABRB3_P92_F methylation site for Homo sapiens gamma-aminobutyric acid (GABA) A receptor, beta 3 (GABRB3)
cttccagcccctgccgtggcggccctatttttcatttatacaattggacct

SEQ ID NO: 22 - DNA, 45 bp, Homo sapiens
  misc_feature: GRB10_P496_R methylation site for Homo sapiens growth factor receptor-bound protein 10 (GRB10)
tactctgtcgtgggctgaaggcacccggcctgggaaaaggaaacc

SEQ ID NO: 23 - DNA, 56 bp, Homo sapiens
  misc_feature: HDAC9_P137_R methylation site for Homo sapiens histone deacetylase 9 (HDAC9)
gcattaatgcaggctccaatcactcggccatgcttgacctatttttggctcaggcc

SEQ ID NO: 24 - DNA, 59 bp, Homo sapiens
  misc_feature (28)..(29): HIC-1_seq_48_S103_R methylation site for HIC-1_seq_48_S103_R
tagtctcctctatcgctggatgaagcacgagccgggcctgggtagctatggcgacgagc

SEQ ID NO: 25 - DNA, 49 bp, Homo sapiens
  misc_feature: HOXA11_P698_F methylation site for Homo sapiens homeo box A11 (HOXA11)
tcattcatggtcacttccgaagcgctttagtgccttccgtccctaaacc

SEQ ID NO: 26 - DNA, 54 bp, Homo sapiens
  misc_feature: HS3ST2_E145_R methylation site for Homo sapiens heparan sulfate (glucosamine) 3-O-sulfotransferase 2 (HS3ST2)
cgcaggctgctcttcgccttcacgctctcgctctcctgcacttacctgtgttac

SEQ ID NO: 27 - DNA, 50 bp, Homo sapiens
  misc_feature: HTR1B_E232_R methylation site for Homo sapiens 5-hydroxytryptamine (serotonin) receptor 1B (HTR1B)
ggtagttagccggggtgtgcagtttccgggtccggtacactgtggcaatc

SEQ ID NO: 28 - DNA, 52 bp, Homo sapiens
  misc_feature: HTR1B_P222_F methylation site for Homo sapiens 5-hydroxytryptamine (serotonin) receptor 1B (HTR1B)
cttccagagcgcctagctaagccgccgcgtctgtggttgttcctctccacac

SEQ ID NO: 29 - DNA, 48 bp, Homo sapiens
  misc_feature (25)..(26): IL1B_P582_R methylation site for Homo sapiens interleukin 1, beta (IL1B)
ttcttggctggggcagagaacatacggtatgcagggttcaggctcctg

SEQ ID NO: 30 - DNA, 46 bp, Homo sapiens
  misc_feature: IL6_E168_F methylation site for Homo sapiens interleukin 6 (interferon, beta 2) (IL6)
gtgtggcccagggagggctggcgggcggccagcagcagaggcaggc

SEQ ID NO: 31 - DNA, 53 bp, Homo sapiens
  misc_feature (22)..(23): KIAA1804_P689_R methylation site for Homo sapiens mixed lineage kinase 4 (KIAA1804)
gcactggcccaggtctggcaccgcgctacaatttcttctgtagcccgttctga

SEQ ID NO: 32 - DNA, 49 bp, Homo sapiens
  misc_feature: LMO2_P794 methylation site for Homo sapiens LIM domain only 2 (rhombotin-like 1) (LMO2)
ctgtctgctgggcaaggcccaattccgaggtgacagctcaccgggcctc

SEQ ID NO: 33 - DNA, 54 bp, Homo sapiens
  misc_feature: LOX_P313_R methylation site for Homo sapiens lysyl oxidase (LOX)
aggcgaaggcagccaggccatggggcgacgccaaaatatgcacgaagaaaaatg

SEQ ID NO: 34 - DNA, 44 bp, Homo sapiens
  misc_feature: MATK_P190_R metylation site for Homo sapiens megakaryocyte-associated tyrosine kinase (MATK)
ctcccggggcataaggaaggaagcggggctgcaggtaccgcctg

SEQ ID NO: 35 - DNA, 54 bp, Homo sapiens
  misc_feature (29)..(30): MEST_E150_F methylation site for sapiens mesoderm specific transcript homolog (mouse) (MEST)
tcaggaagcgcatgcgcaaccggttctccgaaacatggagtcctgtaggcaagg

SEQ ID NO: 36 - DNA, 51 bp, Homo sapiens
  misc_feature (24)..(25): MFAP4_P10_R methylation site for Homo sapiens microfibrillar-associated protein 4 (MFAP4)
tgctcagagtggctgggtgtctgcggccccagactgcaaccgcccagagtt

SEQ ID NO: 37 - DNA, 58 bp, Homo sapiens
  misc_feature (27)..(28): MFAP4_P197_F methylation site for Homo sapiens microfibrillar-associated protein 4 (MFAP4)
gaccacctgtgtctcattagtcctgtcgggcaaagtactgcagacgttaactccctgc

SEQ ID NO: 38 - DNA, 49 bp, Homo sapiens
  misc_feature (23)..(24): MMP14_P13_F methylation site for Homo sapiens matrix metallopeptidase 14 (membrane-inserted) (MMP14)
agggagggaccagaggagagagcgagagagggaaccagaccccagttcg

SEQ ID NO: 39 - DNA, 50 bp, Homo sapiens
  misc_feature (21)..(22): MMP7_E59_F methylation site for Homo sapiens matrix metallopeptidase 7 (matrilysin, uterine) (MMP7)
caggcacacagcacacagcacggtgagtcgcatagctgccgtccagagac

SEQ ID NO: 40 - DNA, 50 bp, Homo sapiens
  misc_feature (29)..(30)
  misc_feature (29)..(30): MST1R_E42_R methylation site for Homo sapiens macrophage stimulating 1 receptor (c-met-related tyrosine kinase) (MST1R)
agcagcaacaggaaggactgaggcagcggcgggaggagctccatcgaggc

SEQ ID NO: 41 - DNA, 52 bp, Homo sapiens
  misc_feature (27)..(28): NOS2A_E117_R methylation site for Homo sapiens nitric oxide synthase 2A (inducible, hepatocytes) (NOS2A)
ggaagagacctgtgccttgagaacttcgggactgtctagaactgcccagtcc

SEQ ID NO: 42 - DNA, 50 bp, Homo sapiens
  misc_feature (24)..(25): NOTCH1_P1198_F methylation site for sapiens Notch homolog 1, translocation-associated (Drosophila) (NOTCH1)
caaaatgcctgccatagtccctgcgcaaagttcacggcctcgtgccaggg

SEQ ID NO: 43 - DNA, 54 bp, Homo sapiens
  misc_feature (23)..(24): NOTCH4_E4_F methylation site for Homo sapiens Notch homolog 4 (Drosophila) (NOTCH4)
cctcggcctgctgcaagcctcacgtctgagctgtttcctgagtcacacaatgtc

SEQ ID NO: 44 - DNA, 44 bp, Homo sapiens
  misc_feature (24)..(25): NPR2_P1093_F methyltion site for Homo sapiens natriuretic peptide receptor B/guanylate cyclase B (atrionatriuretic peptide receptor B) (NPR2)
aggacaaaccctggggtcgctggcgtgtgtgagatggaaatgga

SEQ ID NO: 45 - DNA, 50 bp, Homo sapiens
  misc_feature (27)..(28): NQO1_P345_R methylation site for Homo sapiens NAD(P)H dehydrogenase, quinone 1 (NQO1)
aaatggagcagaaaaagagccggatgcggattactgtggtgccctaggct

SEQ ID NO: 46 - DNA, 54 bp, Homo sapiens
  misc_feature (26)..(27): NRG1_P558_R methylation site for Homo sapiens neuregulin 1 (NRG1)
agcgcaacctagcatctttaaggttcgcttagcccttcctgtgcacctggaagg

SEQ ID NO: 47 - DNA, 47 bp, Homo sapiens
  misc_feature (21)..(22): PALM2-AKAP2_P183_R methylation site for Homo sapiens PALM2-AKAP2 protein (PALM2-AKAP2)
ggtccatcacactccaggggcggagcgaggcaccgagacgtcagggc

SEQ ID NO: 48 - DNA, 52 bp, Homo sapiens
  misc_feature (27)..(28): PECAM1_P135_F methylation site for Homo sapiens platelet/endothelial cell adhesion molecule (CD31 antigen) (PECAM1)
caaggcacaagtgacatttgccttggcgttcttgaccctccctctgtctcgc

SEQ ID NO: 49 - DNA, 47 bp, Homo sapiens
  misc_feature (21)..(22): PLAT_E158_F methylation site for Homo sapiens plasminogen activator, tissue (PLAT)
gcttgctccttccctttcctcgcagaggttttctctccagccctgga

SEQ ID NO: 50 - DNA, 55 bp, Homo sapiens
  misc_feature (28)..(29): PLS3_E70_F methylation site for Homo sapiens plastin 3 (T isoform) (PLS3)
ggcagtcgggccagacccaggactctgcgactttacgtaagtgctttgtaggcgc

SEQ ID NO: 51 - DNA, 45 bp, Homo sapiens
  misc_feature (23)..(24): PRKCDBP_E206_F methylation site for Homo sapiens protein kinase C, delta binding protein (PRKCDBP)
gcccaggccgctctggatgcggcgcacggaccctgccaggcctcc

SEQ ID NO: 52 - DNA, 50 bp, Homo sapiens
  misc_feature (22)..(23): RAB32_P493_R methylation site for Homo sapiens RAB32, member RAS oncogene family (RAB32)
agcccagtgttatccgtccttcgttaagttcaaagtcacggtgccacttc

SEQ ID NO: 53 - DNA, 44 bp, Homo sapiens
  misc_feature (20)..(21): RARA_P1076_R methylation site for Homo sapiens retinoic acid receptor, alpha (RARA)
cctctcccctcaagtctgtcgctgacttcctctggcccttcccc

SEQ ID NO: 54 - DNA, 48 bp, Homo sapiens
  misc_feature (24)..(25): RBP1_E158_F methylation site for Homo sapiens retinol binding protein 1, cellular (RBP1)
gcgcaggtactcctcgaaattctcgttgaccaacatcttccagtaccc

SEQ ID NO: 55 - DNA, 49 bp, Homo sapiens
  misc_feature (27)..(28): SCGB3A1_E55_R methylation site for Homo sapiens secretoglobin, family 3A, member 1 (SCGB3A1)
ctcaccggagctgcaggacagggccacgcagagccccaggagggcggcg

SEQ ID NO: 56 - DNA, 54 bp, Homo sapiens
  misc_feature (27)..(28)
  misc_feature (27)..(28): SEPT5_P464_R methylation site for Homo sapiens septin 5 (SEPT5)
cctacagcctgccaggtgcgtctgctcgcagagcaggtctgcgcagcaccgagc

SEQ ID NO: 57 - DNA, 48 bp, Homo sapiens
  misc_feature (28)..(29): SLC5A5_E60_F methylation site for Homo sapiens solute carrier family 5 (sodium iodide symporter), member 5 (SLC5A5)
ggacagacagccggctgcatgggacagcggaacccagagtgagagggg

SEQ ID NO: 58 - DNA, 49 bp, Homo sapiens
  misc_feature (26)..(27): SLC5A8_E60_R methylation site for Homo sapiens solute carrier family 5 (iodide transporter), member 8 (SLC5A8)
actggagtggccgagttcgccaaggcgccggggacacctgagcagatga

SEQ ID NO: 59 - DNA, 50 bp, Homo sapiens
  misc_feature (25)..(26): SOX1_P294_F methylation site for Homo sapiens SRY (sex determining region Y)-box 1 (SOX1)
gggccgggcccagcgcaccgctcccggccccaaaagcggagctgcaactt

SEQ ID NO: 60 - DNA, 52 bp, Homo sapiens
  misc_feature (23)..(24): SPARC_P195_F methylation site for Homo sapiens secreted protein, acidic, cysteine-rich (osteonectin) (SPARC)
accctgcctgcctcatctgttccggggctgctgcctaaaccgactcacagag

SEQ ID NO: 61 - DNA, 47 bp, Homo sapiens
  misc_feature (22)..(23): SPI1_P48_F methylation site for Homo sapiens spleen focus forming virus (SFFV) proviral integration oncogene spi1 (SPI1)
gtccccttggggtgacatcaccgccccaacccgtttgcataaatctc

SEQ ID NO: 62 - DNA, 46 bp, Homo sapiens
  misc_feature (22)..(23): TDGF1_P428_R methylation site for Homo sapiens teratocarcinoma-derived growth factor 1 (TDGF1)
acacacacctagctcctcaggcggagagcacccctttcttggccac

SEQ ID NO: 63 - DNA, 51 bp, Homo sapiens
  misc_feature (27)..(28): TEK_P479_R methylation site for Homo sapiens TEK tyrosine kinase, endothelial (venous malformations, multiple cutaneous and mucosal) (TEK)
gcttttcaggttgtattttctcatcacggaaaccttcttctcccaattcaa

SEQ ID NO: 64 - DNA, 52 bp, Homo sapiens
  misc_feature (23)..(24): TNFRSF10C_P612_R methylation site Homo sapiens tumor necrosis factor receptor superfamily, member 10c, decoy without an intracellular domain (TNFRSF10C)
ctcctcagcctctgcatgtgcccgtcatggcccctgtgtccttcattctgtc

SEQ ID NO: 65 - DNA, 51 bp, Homo sapiens
  misc_feature (20)..(21): TPEF_seq_44_S36_F methylation site for Homo sapiens transmembrane protein with EGF-like and two follistatin- like domains 2 (TMEFF2)
agcagccagcaaaagccctcgcaaagtgtccagctgctgcactgccgcggg

SEQ ID NO: 66 - DNA, 54 bp, Homo sapiens
  misc_feature (28)..(29): TRIP6_P1274_R methylation site for Homo sapiens thyroid hormone receptor interactor 6 (TRIP6)
cttgggcatggtgcccgcttggcatagcgcccggctccggatcttcctgtgcct

SEQ ID NO: 67 - DNA, 51 bp, Homo sapiens
  misc_feature (21)..(22): TUSC3_E29_R methylation site for Homo sapiens tumor suppressor candidate 3 (TUSC3)
caggtcttctcccggtgaaccggatgctctgtcagtctcctcctctgcgtc

SEQ ID NO: 68 - DNA, 52 bp, Homo sapiens
  misc_feature (28)..(29): WNT2_E109_R methylation site for Homo sapiens wingless-type MMTV integration site family member 2 (WNT2)
aaagtttcaaacgatgggcccagcgagcgataaaggccagcccggaccgcct

SEQ ID NO: 69 - DNA, 49 bp, Homo sapiens
  misc_feature (24)..(25): WNT2_P217_F methylation site for Homo sapiens wingless-type MMTV integration site family member 2 (WNT2)
agagcatccgtgggctctcggagcgtgcgttccggattgccgaggccat

SEQ ID NO: 70 - DNA, 55 bp, Homo sapiens
  misc_feature (28)..(29): ZMYND10_P329_F methylation site for Homo sapiens zinc finger, MYND-type containing 10 (ZMYND10)
atggcttcttggttcctctatttctcgcgtcccggctccactagttggctcctga

SEQ ID NO: 71 - DNA, 3267 bp, Homo sapiens
ggtcaccgcccggctgcggggccagtggcaggagcgccacgcaccgccagccgcagggggcgtgggatgggggcggccggggaggggggcgcccacactgactagagccaaccgcgcacttcaaaagggtgtcggtgccgcgctcccctcccgcggcccgggaacttcaaagcgggccgtgctgccccggctgcctcgctctgctctggggcctcgcagccccggcgcggccgcctggtggcgatgacccgggcgctctgctcagcgctccgccaggctctcctgctgctcgcagcggccgccgagctctcgccaggactgaagtgtgtatgtcttttgtgtgattcttcaaactttacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggagctggccatcattattactgtgcctgtttgcctcctgtccatagctgcgatgctgacagtatgggcatgccagggtcgacagtgctcctacaggaagaaaaagagaccaaatgtggaggaaccactctctgagtgcaatctggtaaatgctggaaaaactctgaaagatctgatttatgatgtgaccgcctctggatctggctctggtctacctctgttggttcaaaggacaattgcaaggacgattgtgcttcaggaaatagtaggaaaaggtagatttggtgaggtgtggcatggaagatggtgtggggaagatgtggctgtgaaaatattctcctccagagatgaaagatattggtttcgtgaggcagaaatttaccagacggtcatgctgcgacatgaaaacatccttggtttcattgctgctgacaacaaagataatggaacttggactcaactttggctggtatctgaatatcatgaacagggctccttatatgactatttgaatagaaatatagtgaccatggctggaatgatcaagctggcgctctcaattgctagtggtctggcacaccttcatatggagattgttggtacacaaggtaaacctgctattgctcatcgagacataaaatcaaagaatatcttagtaaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcctaatgatgataattatgttaaaaagaaatctctcatagctttcttttccattttcccctttatgtgaatgtttttgccattttttttttgttctacctcaaagataagacagtacagtatttaagtgcccataaggcagcatgaaaagataactctaaagttaagcatgggcaggagttgacttcatccaatctctatgttatgtttaattttattttgaaagcaacacctcaactcatctttttatttaataaggaagaaatatattacaaaagtataaaataagctctataaaaatgttatagtcattaagtttttattttacttgaaccaagagcacatgaatgaacaggaaaagatgtaaaaacatttttttctgagatgaaaacatattaattaaacatgcaaattagagcatgctatctttaggtgatgcaatctatgtttccccctttttaagttagcaggactttttaaaaataaatattgctctaaactttaatatatcgaacgtgagagtggagctgcttagtggaagatgtaagtgaggtgggtgtcccatgtgcttggtctccccttctgctgttctcctgttcttcataatccactactgcagcagtccctgaaccactaaacttgttcctttcatttacaaaagagatacctgacatcctgagacactgagaaatgtcctgaagtcacacagctaatggcagaactggcactaggtccaaatcttgtgataatgaacaccgtaaggttagctagcttcctactttcccttgaatagtgcttttctccctatgtaatatcttttattatgatatttgtggtttagaaggcatattgagttattttgcagaatcataatggacccgcacaaaatctcagaaccatatctgttgacattttttctcatagaaatatcatggttaccccatttgttaatgagcattaatgttttctgaacacttccaaagattaatcaaacataaatattcattgtctgaaaatgtctttaagatacaattcagaggtccctatttcctttgtacatacacacttagaaagaaaagacagaaaaggaagaggaaggaaggaaatattttgagaatatattgagaagaattaagaaaactcttcaatgaagtgttaacaaccaaaccctacagacggtatcagaaacagcaaatagatattcctctaccctttcacagtgagtgagtgagtacagaagaatgctcatgatagttttgccttcattctactttctgtggacacagagtaatgaatatttaatgggacattaaatatgcccttcaaatctataattttactttggtaaacgagatttaacatgatgtcttttatgctcctaaaacatcttttttcaaactccattccttagaacattcttctactgagatgatccaagaccaaaagtgttctttggtacttgcttataaagtgatagtacatgttagcatataatgtattttgaagagtgaagtaaatgctattgataacagaaaaaaaaaaaaaaaa

SEQ ID NO: 72 - DNA, 6767 bp, Homo sapiens
catggatcctttggattttgattccagttgatccctggagtaaggtcctaaccggggtctcccgaggtcgtttcgccgtccaggatggagcaggcggggagctcgcaccgccgcgcccgggccgcgagtgatgataacctaagaggccggcgcgggcgggcgtgagcggcggaggagccgggcgcggcgacacgcggccatggagcgggagccggcggggaccgaggagcccgggcctccgggacggcggaggcgccgagagggcaggacgcgcacggtgcgctccaacctgctgccgcccccgggcgccgaggaccctgcggctggcgcggccaagggcgagcggcgacggcggcgcgggtgtgcccagcacctggccgacaaccggctcaagactaccaagtacacgctgctgtccttcctgcccaagaacctgttcgagcagttccaccgcccggccaacgtgtactttgtcttcatcgcgctgctcaacttcgtgccggcggtgaacgccttccagcccggcctggcactggcgccggtgctcttcatcctggccatcacggccttcagggacctgtgggaggactacagccgccaccgctccgaccacaagatcaaccacctgggctgcctggtcttcagcagggaagaaaagaaatacgtgaaccgattctggaaagaaatccacgtgggagactttgtgcgtcttcgctgcaacgaaatcttccctgcggacattctgctgctctcctccagtgaccccgacgggctatgccacatcgagaccgccaacctggatggagagaccaacctgaagcggcggcaggtggtccgcggcttctcggagcttgtctccgaattcaatcctttgacgttcaccagcgtgatcgaatgcgagaagccaaacaacgacctgagtaggtttcgcggctgcatcatacatgacaacgggaaaaaggccgggctgtataaagaaaacctgctgctgaggggctgcacccttaggaacacggacgcagtcgtcggcattgtcatctacgcaggacatgaaaccaaggctctgctgaacaacagtgggccccgctacaagcgcagcaagctggagaggcagatgaactgcgacgtgctctggtgtgtcctgctccttgtttgcatgtctctgttttcagcagtcggacatggactgtggatatggcggtatcaagagaagaagtcattattttatgtccccaagtctgatggaagctccttatccccagtcacagctgcagtttactcatttttaacaatgataatagttctgcaggttttgatcccaatttccttatacgtttccattgaaattgttaaagcatgccaagtgtacttcattaaccaggacatgcagttgtatgacgaagaaacagactcgcagctgcagtgccgagctctgaacatcacggaagacttaggacagatacagtacattttctcagataaaactggcactttgacagagaataagatggttttccgaagatgcactgtgtctggtgtagaatattctcatgatgcaaatgcgcagcgtctggccaggtaccaagaggcagactcggaggaggaggaggtggtgcccagagggggctcggtgtcccagcgcggcagcatcggcagccaccagagtgtccgggtggtgcacagaacccagagcaccaagtcccaccggcgcacgggcagccgggccgaggccaagagggccagcatgctgtccaagcacacggccttcagcagccccatggagaaggatatcacgcccgacccaaagctgctggagaaggtgagtgagtgtgacaagagcctagccgtggcgaggcatcaggagcacctgctggcccacctctcgcccgagctgtctgacgtctttgatttcttcatcgcactcaccatctgcaacacagtcgtcgtcacgtccccggatcagccacgaacaaaggtgagggtgaggtttgagctgaagtccccggtgaagacgatagaagacttcctgcggaggttcacacccagctgcctgacctcaggctgcagcagcatcgggagcctggccgccaacaagtccagccacaagttgggctccagcttcccgtccaccccgtccagcgacggcatgcttctcaggctggaggagaggctgggccagcccacctcggccatcgccagcaacggctacagcagccaggcggacaactgggcctcggagcttgctcaggagcaggagtcagagcgcgagctgcggtacgaggcggagagcccggatgaggccgcactggtgtatgcggccagagcctacaactgcgtgcttgtggagcggctgcacgaccaagtgtcagtggagctgccccacctgggcaggctcaccttcgagctcctgcacacactgggtttcgattccgtccgcaagaggatgtcagtggtgatccggcacccgcttaccgatgagatcaacgtctacaccaagggggccgactcagtggtcatggatctcctgcagccctgctcttcagttgacgccagagggaggcatcaaaaaaagattcggagcaaaactcagaattacctcaacgtgtatgcggcggaaggcctgcgcaccttgtgcatcgccaagagagttctgagtaaagaagagtatgcctgctggttgcaaagccacctagaagccgaatcctccctggaaaacagcgaggagctcctcttccagtctgccattcgcctggagaccaacctgcacttgttaggtgccactgggattgaagaccgcctgcaggacggagtccctgaaactatttctaaattgcgtcaagcgggcctgcagatttgggttctcactggtgacaaacaagaaacagctgtcaacattgcatatgcctgcaaactgctggaccacgacgaggaggtcatcaccctgaatgccacctcccaggaggcgtgtgcagccctgctagaccagtgcctatgctacgtgcagtccagaggcctccagagagcccctgagaagaccaagggcaaagtgagcatgaggttctcctctctctgcccaccctccacgtccactgcctctggccgcagacccagcctcgtgatcgatgggagaagcctggcctacgctctcgagaaaaacctggaggacaaattcctcttccttgccaagcagtgccgctccgtcctctgctgtcggtcgacgcctctgcagaagagcatggtggtgaagctggtgcggagcaagctcaaggccatgaccctggccataggtgatggagccaatgatgtcagcatgatccaggtggcagatgtgggtgtgggaatctccggccaggagggtatgcaggcagtgatggccagcgactttgcagtgccgaaattccgatacctggagaggctcttgattcttcacgggcattggtgctactcccgacttgccaacatggtgctgtacttcttctacaaaaacacaatgttcgtgggcctcctgttttggttccagtttttctgtggcttctctgcatctaccatgattgaccagtggtatctaatcttctttaatctgctcttctcgtcacttcccccgctcgtgactggggtgctggacagggatgtgccagccaatgtgctgctgaccaacccgcagctctacaagagtggccagaacatggaggaataccggccacgaacgttctggtttaacatggccgacgccgccttccagagcctggtttgcttttccattccttacctggcctactatgactcgaacgtggacctgtttacctgggggacccctattgtgacaatcgcgctgctcactttcctgctccacctgggcattgaaaccaaaacctggacctggctcaactggataacgtgtggcttcagtgtccttttgtttttcaccgtggctttgatttacaatgcgtcttgtgccacgtgctatcctccgtccaacccttactggactatgcaagccttactgggtgacccagtgttttacttgacttgcctgatgacgcctgtcgctgcactgctgcccagattgtttttcagatccctccaggggagggttttccccacacaacttcagctggcacgtcagttgaccaggaagtcccccaggagatgcagtgctcccaaagagacctttgctcagggacgcctcccgaaggactcgggaaccgagcactcatcagggaggacagtcaagacctctgtgcccctgtcccagccttcttggcacacacagcagccggtctgctccctggaggccagcggggagcccagcacagtggacatgagcatgccagtgagggagcacaccctgctggaggggctgagcgcaccggcccccatgtcctctgcgccaggggaggctgtcctgaggagtccaggagggtgtcctgaggagtccaaggtgagagctgccagcaccggcagggtgacccccctgtcttccctcttcagcctgcctaccttcagcttactcaactggatttcctcctggtcgctggtcagcaggctggggagtgtcttacagttctcccggacggagcagcttgcagatggacaagcgggacgtggacttcctgtccagccccactcaggccgatcaggacttcaagggccagaccacagactacttataggagcatcttcaaggcggtcacagtgaaaaccttgaaatggccttttttaatatatataaataaatgttaatattatttatgtttattatttgcacagaagagttctagggagatgtatttctaaatgtttcccaggctaatacaggaaacaagaggtaccaaaaaagaaagtttattttttaaaattctaagtagagtatattgaaaagaaaaagaagagccttaacatatataaaagtttaaagaagagtaacacttgaaaagtgtgtttagatttattttttcatctcatttttaagaacaagcagtacgatttgttttcttcaacatgtgtgactgcgcactgagtacaaatgtgtgactgctcatggttaatgcaggcaggtgtgaacatgggggaacaatgagcagagatggcagagggcagagcacatggcccccagaggcttccagtctcactgacacaggagggctgggctccacttcatccagatgaaggaaaggaagacctcaagaaaaattcacagttgagtgcatcccagcattctgttccgggcaggcatttcaggaagaccgccttgtaggtattacatccctggtgtcgtattttgcctgttaaatcgtaacaagcaataaacaactttcactttgcaaagacagtgtgtccagttaccactggtgtatgaaatgattaatacctgacctcacagagtatgatctgagggcacttccgtaaggcaagtccttttagaggctatgaagaaaacagctgcatggcacataccaaagctgctgcacagccggccaccatggcaccctgcaccaggccatcagcaccacgtgccaaggagctcagcggtcttcaggcatttttgtaatgagccattagttctgtccctctaaaactagaaaaggaagggcaggaaatgataacaacccaaggcaatgatatggcatgtcatcttctgagcccttctttctactttgtcaaacagttcttagttgctggctctgctcggcaccggggctgtgaagggtgtactccctgctgtgtgggagggacctagggcctctttggatgctgtcttcgaggacagcaatgcagagagggcataggatctgaggacaaggaaattcctcagcatggcgtatcaggaaagcatggctcattctgcaatgagccatgagtgtgggccatcgcaagtcacagaaattgcacctcattccagtcaagcagaaaaacaggcacaggctcagtgtaggtcccaagagagggtgcctggactcagcaactcggacctgggcttttctcccagctttcagggacagctttgtcctgagtctgcctctgttcacggggatgcttggctggagtcacccccaggacttatccatgcatcactattcagaagacacagagggcccctctctccacattccaaacagagtcctggtttcctcagcctcaccctgcatagcttgcacaacatcctcagaaccattcactggcaaatggaggggaacgtgctgactgggactcccagctggagctgggaggagaggtccacttcccttagaacacctgagctgctgcatgagtggacgtcagaagaatctctatgccctgttaaatggggagacaaaggggtggtgggggcttcagccagtgatttcggaccgaaggtgacagccgtcccaaccctgcccagcctgatgccacctcctctgttcttggaacaacgcataggaaaagaatctcctttggaaggtgacactgctccctgaattaaggtaatggttgcgagcaccaagtacaaggactagacgcatatttacctgcgtatctgagagttccagattcccagcttccagatgatccttgcacagacaacctaccttctttccagaggatgtctttctcctctggagagtagatgcttgctcttgggaaacggaatgaccttggcgctggcttcaggaatatgcatcccacagccagtttagagaaatacatgttgtaaatggcattgacagctgctctttaggatggggagtattatggaaatccacaataacaatctatggcaagcaact

SEQ ID NO: 73 - DNA, 3655 bp, Homo sapiens
cttcagatagattatatctggagtgaaggatcctgccacctacgtatctggcatagtattctgtgtagtgggatgagcagagaacaaaaacaaaataatccagtgagaaaagcccgtaaataaaccttcagaccagagatctattctccagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttggtttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatataggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctttctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttcta

SEQ ID NO: 74 - DNA, 1173 bp, Homo sapiens
atggaggaaccgggtgctcagtgcgctccaccgccgcccgcgggctccgagacctgggttcctcaagccaacttatcctctgctccctcccaaaactgcagcgccaaggactacatttaccaggactccatctccctaccctggaaagtactgctggttatgctattggcgctcatcaccttggccaccacgctctccaatgcctttgtgattgccacagtgtaccggacccggaaactgcacaccccggctaactacctgatcgcctctctggcggtcaccgacctgcttgtgtccatcctggtgatgcccatcagcaccatgtacactgtcaccggccgctggacactgggccaggtggtctgtgacttctggctgtcgtcggacatcacttgttgcactgcctccatcctgcacctctgtgtcatcgccctggaccgctactgggccatcacggacgccgtggagtactcagctaaaaggactcccaagagggcggcggtcatgatcgcgctggtgtgggtcttctccatctctatctcgctgccgcccttcttctggcgtcaggctaaggccgaagaggaggtgtcggaatgcgtggtgaacaccgaccacatcctctacacggtctactccacggtgggtgctttctacttccccaccctgctcctcatcgccctctatggccgcatctacgtagaagcccgctcccggattttgaaacagacgcccaacaggaccggcaagcgcttgacccgagcccagctgataaccgactcccccgggtccacgtcctcggtcacctctattaactcgcgggttcccgacgtgcccagcgaatccggatctcctgtgtatgtgaaccaagtcaaagtgcgagtctccgacgccctgctggaaaagaagaaactcatggccgctagggagcgcaaagccaccaagaccctagggatcattttgggagcctttattgtgtgttggctacccttcttcatcatctccctagtgatgcctatctgcaaagatgcctgctggttccacctagccatctttgacttcttcacatggctgggctatctcaactccctcatcaaccccataatctataccatgtccaatgaggactttaaacaagcattccataaactgatacgttttaagtgcacaagttga

SEQ ID NO: 75 - DNA, 4667 bp, Homo sapiens
cagggcctgggcacgaccatggtgggacgtcgcccgcggcttcggggaccgctgcggcagcagaggcggctggccaggaacgcgggccgaggctggaccctttgggcagctagcccgtgatctctgccgtcaccgatcgcgattcctaccccctcgccttcccccggcgccgacggccacaccgccggacgatgcgcgcccgcggccgcccgggaggctgagcccagcttcccgctccgccttccccgcgcagctgcccccatggctttgcggggcgccgcgggagcgaccgacaccccggtgtcctcggccgggggagcccccggcggctcagcgtcctcgtcgtccacctcctcgggcggctcggcctcggcgggcgcggggctgtgggccgcgctctatgactacgaggctcgcggcgaggacgagctgagcctgcggcgcggccagctggtggaggtgttgtcgcaggacgccgccgtgtcgggcgacgagggctggtgggcaggccaggtgcagcggcgcctcggcatcttccccgccaactacgtggctccctgccgcccggccgccagccccgcgccgccgccctcgcggcccagctccccggtacacgtcgccttcgagcggctggagctgaaggagctcatcggcgctgggggcttcgggcaggtgtaccgcgccacctggcagggccaggaggtggccgtgaaggcggcgcgccaggacccggagcaggacgcggcggcggctgccgagagcgtgcggcgcgaggctcggctcttcgccatgctgcggcaccccaacatcatcgagctgcgcggcgtgtgcctgcagcagccgcacctctgcctggtgctggagttcgcccgcggcggagcgctcaaccgagcgctggccgctgccaacgccgccccggacccgcgcgcgcccggcccccgccgcgcgcgccgcatccctccgcacgtgctggtcaactgggccgtgcagatagcgcggggcatgctctacctgcatgaggaggccttcgtgcccatcctgcaccgggacctcaagtccagcaacattttgctacttgaagagatagaacatgatgacatctgcaataaaactttgaagattacagattttgggttggcgagggaatggcacaggaccaccaaaatgagcacagcaggcacctatgcctggatggcccccgaagtgatcaagtcttccttgttttctaagggaagcgacatctggagctgtggagtgctgctgtgggaactgctcaccggagaagtcccctatcggggcattgatggcctcgccgtggcttatggggtagcagtcaataaactcactttgcccattccatccacctgccctgagccgtttgccaagctcatgaaagaatgctggcaacaagaccctcatattcgtccatcgtttgccttaattctcgaacagttgactgctattgagggggcagtgatgactgagatgcctcaagaatcttttcattccatgcaagatgactggaaactagaaattcaacaaatgtttgatgagttgagaacaaaggaaaaggagctgcgatcccgggaagaggagctgactcgggcggctctgcagcagaagtctcaggaggagctgctaaagcggcgtgagcagcagctggcagagcgcgagatcgacgtgctggagcgggaacttaacattctgatattccagctaaaccaggagaagcccaaggtaaagaagaggaagggcaagtttaagagaagtcgtttaaagctcaaagatggacatcgaatcagtttaccttcagatttccagcacaagataaccgtgcaggcctctcccaacttggacaaacggcggagcctgaacagcagcagttccagtcccccgagcagccccacaatgatgccccgactccgagccatacagttgacttcagatgaaagcaataaaacttggggaaggaacacagtctttcgacaagaagaatttgaggatgtaaaaaggaattttaagaaaaaaggttgtacctggggaccaaattccattcaaatgaaagatagaacagattgcaaagaaaggataagacctctctccgatggcaacagtccttggtcaactatcttaataaaaaatcagaaaaccatgcccttggcttcattgtttgtggaccagccagggtcctgtgaagagccaaaactttcccctgatggattagaacacagaaaaccaaaacaaataaaattgcctagtcaggcctacattgatctacctcttgggaaagatgctcagagagagaatcctgcagaagctggaagctgggaggaggcagcctctgcgaatgctgccacagtcaccattgagatggctcctacgaatagtctgagtagatccccccagagaaagaaaacggagtcagctctgtatgggtgcaccgtccttctggcatcggtggctctgggactggacctcagagagcttcataaagcacaggctgctgaagaaccgttgcccaaggaagagaagaagaaacgagagggaatcttccagcgggcttccaagtcccgcagaagcgccagtcctcccacaagcctgtcatccacctgtggggaggccagcagcccaccctccctgccactgtcaagtgccctgggcatcctctccacaccttctttctccacaaagtgcctgctgcagatggacagtgaagatccactggtggacagtgcacctgtcacttgtgactctgagatgctcactccggatttttgtcccactgccccaggaagtggtcgtgagccagccctcatgccaagacttgacactgattgtagtgtatcaagaaacttgccgtcttccttcctacagcggacatgtgggaatgtaccttactgtgcttcttcaaaacatagaccatcacatcacagacggaccatgtctgatggaaatccgaccccaactggtgcaactattatctcagccactggagcctctgcactgccactctgcccctcacctgctcctcacagtcatctgccaagggaggtctcacccaagaagcacagcactgtccacatcgtgcctcagcgtcgccctgcctccctgagaagccgctcagatctgcctcaggcttacccacagacagcagtgtctcagctggcacagactgcctgtgtagtgggtcgcccaggaccacatcccacccaattcctcgctgccaaggagagaactaaatcccatgtgccttcattactggatgttgacgtggaaggtcagagcagggactacactgtgccactgggtagaatgaggagcaaaaccagccggccatctatatatgaactggagaaagaattcctgtcttaaactaagtgccttactgttgtttaagcatttttttaaggtgaacaaatgaacacaatgtgtctacctttgaactgtttcatgctgctgtgttttcaaaagctgtggccatgttcctaaattagtaagatatatccagcttctcaaaaaatgtatatgattgctgttagccatgtctattgtttttcctctggattcttttcttataacttggaatacacaaaagtataaaacaagagatgtgcaccaatgaaaactatgctgggtcgaattaccttcagcacaatgttaatgttttcgttctcatttatgcctttgtccatttgcacacaacagaaattgtaatgagcttcactatttttgtttctttccttccttttttttcttttttcctttctttcctttttcttgtcttgtttcttgtttttttctcttgtagtttcttttcttaattgtcatttttgcaacaaaaagccaagaaagagctttagtttcttggcaagaataatgtgatattagtaagtaaaggttcttaaaagtctgatgactggaatagatataaagtcctgtttaaactacctaaccttggctgtgggccgataatgcatatgtccagttctcacttaaattatgcaatgatatttctctctgaggaaattatacggaatgtaacttataaaagctttactgaatataagttataagcattttattcattagaactccaaaatagatgttcaaagttcagtccttgccatttgactgagaccacatggtgtgccccttgagtgaggctaatctttaggtttttcctatagaaaacgttcttcctccatcagtagccctttatttgatattcagaagtggaaagctttttcattctccagtagaacttttaaaaattgttacagatacctagctcttcacagatatcatgtattgtaaacagtcatgtgtcttaattttattttctctatttgagtgcataattatcctaataatcccaaagacactgacaactcaaggaacagcagtacagtactattagaagttaagtatgttgttgttatttcacatttcatttaattgtggataaatgttagacatctgttgaaataagctcatatggtggaaacgacaactatattatgaattattttcagaaatggatctttgaatagcagatcaggatttaaataataaaattatctatgaatcacttttatggtcatacatatatgatacaaatccagagttattggtgcagaaatggctacccgagagcttggtaaatttgc

SEQ ID NO: 76 - DNA, 1830 bp, Homo sapiens
agccactctgagcagaactgacagcatgaaggcactcctggccctgccgctgctgctgcttctctccacgcccccgtgtgccccccaggtctccgggatccgaggagatgctctggagaggttttgccttcagcaacccctggactgtgacgacatctatgcccagggctaccagtcagacggcgtgtacctcatctacccctcgggccccagtgtgcctgtgcccgtcttctgtgacatgaccaccgagggcgggaagtggacggttttccagaagagattcaatggctcagtaagtttcttccgcggctggaatgactacaagctgggcttcggccgtgctgatggagagtactggctggggctgcagaacatgcacctcctgacactgaagcagaagtatgagctgcgagtggacttggaggactttgagaacaacacggcctatgccaagtacgctgacttctccatctccccgaacgcggtcagcgcagaggaggatggctacaccctctttgtggcaggctttgaggatggcggggcaggtgactccctgtcctaccacagtggccagaagttctctaccttcgaccgggaccaggacctctttgtgcagaactgcgcagctctctcctcaggagccttctggttccgcagctgccactttgccaacctcaatggcttctacctaggtggctcccacctctcttatgccaatggcatcaactgggcccagtggaagggcttctactactccctcaaacgcactgagatgaaaatccgccgggcctgaagggctggccccctcaggcacctttcctcccctggacacccatggtctccatgagtgctccctctgctgcccctgatgcatgcttctgctgattcccgagcaccaactccttacaagggggccttgtggctctcagccatgccacatccctgtcacacacccagggcatccattcctaagccagacccggctcccctacacctgaagttacactgccagcagttccccaggcctcttccgagaggcacatggttctagcctggacctggctgggctccatgagaatgagttgcctccaccctgtcccaacagctgacagccaggagccactctcccagctgcaggcctttgtggtccatcttgtcctgcttcctcactgtggacccctgtctgggccaccctagtgtgctaagctgagcagtgcagtgtgaacagggcccatggtgtattctaggccacagcccagcactcctctgggctgctctcaaaccatgtcccatcttcagcatccctcccaccaacttactcccctgtggtgagtaccgtggaaccccagcccacctcactatcatactcagcttcccctgatggcccatcccagcccctgaagctctatgccaagaacacagctaccgcacaccaccctgaaacagccacagccaaggtaggcatgcatatgaggtcttccccataccctctgggtgttgagaggtttagccacatgagggagcagaggacaatctctgcagggctgggagtgggtagggactgaaggtctcaataaaccttcagaacctgaatgaactggcttcatacacacaaacatatttgtttatcccccaaatgtaggcacctggctcctccttgctcccctgctgatggtgtcctaccccgaactccaaaaattacacctggagtcaggtgcagaagggaaccttgtatttcacaggcctcattttgatggcaaaaagacagtgtaataataacataataataataaaaatataatactgaaaaggaaaaaaaaaaaaaaaaaa

SEQ ID NO: 77 - DNA, 9312 bp, Homo sapiens
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagcagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgaaaaaaaaaaaaaaa

SEQ ID NO: 78 - DNA, 6762 bp, Homo sapiens
agacgtgaggcttgcagcaggccgaggaggaagaagaggggcagtgggagcagaggaggtggctcctgccccagtgagagctctgagggtccctgcctgaagagggacagggaccggggcttggagaaggggctgtggaatgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatagaagaatacatggtagggaggaattccaaaaatgattacccattaaaaggcaggctggaaggccttcctggttttaagatggatcccccaaaatgaagggttgtgagtttagtttctctcctaaaatgaatgtatgcccaccagagcagacatcttccacgtggagaagctgcagctctggaaagagggtttaagatgctaggatgaggcaggcccagtcctcctccagaaaataagacaggccacaggagggcagagtggagtggaaatacccctaagttggaaccaagaattgcaggcatatgggatgtaagatgttctttcctatatatggtttccaaagggtgcccctatgatccattgtccccactgcccacaaatggctgacaaatatttattgggcacctactatgtgccaggcactgtgtaggtgctgaaaagtggccaagggccacccccgctgatgactccttgcattccctcccctcacaacaaagaactccactgtggggatgaagcgcttcttctagccactgctatcgctatttaagaaccctaaatctgtcacccataataaagctgatttgaagtgttaaaaaaaaaaaaaaaaaa

SEQ ID NO: 79 - DNA, 4108 bp, Homo sapiens
ccggccgtctatgctccaggccctctcctcgcggtgccggtgaacccgccagccgccccgatgtacagcatgatgatggagaccgacctgcactcgcccggcggcgcccaggcccccacgaacctctcgggccccgccggggcgggcggcggcgggggcggaggcgggggcggcggcggcggcgggggcgccaaggccaaccaggaccgggtcaaacggcccatgaacgccttcatggtgtggtcccgcgggcagcggcgcaagatggcccaggagaaccccaagatgcacaactcggagatcagcaagcgcctgggggccgagtggaaggtcatgtccgaggccgagaagcggccgttcatcgacgaggccaagcggctgcgcgcgctgcacatgaaggagcacccggattacaagtaccggccgcgccgcaagaccaagacgctgctcaagaaggacaagtactcgctggccggcgggctcctggcggccggcgcgggtggcggcggcgcggctgtggccatgggcgtgggcgtgggcgtgggcgcggcggccgtgggccagcgcctggagagcccaggcggcgcggcgggcggcggctacgcgcacgtcaacggctgggccaacggcgcctaccccggctcggtggcggcggcggcggcggccgcggccatgatgcaggaggcgcagctggcctacgggcagcacccgggcgcgggcggcgcgcacccgcacgcgcaccccgcgcacccgcacccgcaccacccgcacgcgcacccgcacaacccgcagcccatgcaccgctacgacatgggcgcgctgcagtacagccccatctccaactcgcagggctacatgagcgcgtcgccctcgggctacggcggcctcccctacggcgccgcggccgccgccgccgccgctgcgggcggcgcgcaccagaactcggccgtggcggcggcggcggcggcggcggccgcgtcgtcgggcgccctgggcgcgctgggctctctggtgaagtcggagcccagcggcagcccgcccgccccagcgcactcgcgggcgccgtgccccggggacctgcgcgagatgatcagcatgtacttgcccgccggcgaggggggcgacccggcggcggcagcagcggccgcggcgcagagccggctgcactcgctgccgcagcactaccagggcgcgggcgcgggcgtgaacggcacggtgcccctgacgcacatctagcgccttcgggacgccggggactctgcggcggcgacccacgagctcgcggcccgcgcccggctcccgccccgccccggcgcggcgtggcttttgtacagacgttcccacattcttgtcaaaaggaaaatactggagacgaacgccgggtgacgcgtgtcccccactcaccttccccggagaccctggcgaccgccgggcgctgacaccagacttgggttttagactgaacttcggtgttttcttgagactttttgtacagtatttatcacctacggaggaagcggaaagcgttttctttgctcgaggggacaaaaaagtcaaaacgaggcgagaggcgaagcccacttttgtataccggccggcgcgctcactttcctccgcgttgcttccggacggcgccgaccgccggagcccaagtgacgcggagctcgtcgcatttgttataaatgtagtaaggcaggtccaagcacttacaagttttttgtagttgttaccgctcttttgggttggtttgttaatttatacaaagagattaccaccaccaccccctccttcagacggcggagttatattctgggttttgtaaaactttatgtatctgagcatttccatttttttttttgggttttgtattatttcttgtaaatgcattgtgaaaaattttattttcggcgttgcaatgcggggaggagaagtcagattatgtacatagttttctaaaaagcctttcttctaaaaacgaaaaaagaccccccacccaaaatgtttcgagtcaacaaatttaagagacagagcccattttctccataaatttgtaacatgctatttttatgtgcatgttttatgagttcaaaatgcaatgaggaaatctgacagggaaattatctgtatgaactaaaagtaagggaaccccggggaatgggaggacaggatttttcaaggaacctttttcaatgaaagagaaggaagttaaaacctataggttattttgtagagctgagtgttaatacgggccgagaaataaaagtatcttctgctccggctgtttcactgcggacggctggggctgctgcgcgttaccttgctgcaagcggggcgccttccacctggctgggggtctgcgccacagtttggtccagaggagggaggaggaagggaagaccccagtggtgggaccctggaccaggccatggatgaaggacaaagaccagggcaggtcacgggtttcccaattccccagcaattaagatttcgagcagaatttatctaaatgtgtttcaaggaaacacaatcgctgaaccaaaacgtactgcagccgagccccctccgtccatcctctgcccctccccctggcttctttctcttgggaaaacgggcaaaataattgtgctggattctcacacacacagaaatatcgaccatcaccctcccccgcgtgaactgggatgcaagttgctaaccgatgtgaacgcaaaatgccttgttcattattcctgacgagatcttgaggttgtttgatgctttaaattttttaattatattattttctaggtgtttattggtacattgcagttttttttttgaaatttaaaaatttctgtaaaactttgtcttcaagtaatctgacagcattaaatattgcatttaaaaattatactgtagcaaatacatttaaaaattaatcacaacgttaagatgaaattatatttttggaaaaaaaaaacacttgaagcccagatggaaatacgtttatttcagcagccttaggtttcccctcgctttctcaacacccttccttgtcctggagtatggactgtccgtccaaaagtgagcctatgctataagtttaatgagaaccgaattcagcctgcattcgagaatagctttaagtataatgctgatctgacaattgacgtgtaatttgggaagtcattttgataattttgcttaaaccactcattcgttaaagtgattacaaaaaagttcaagaatgatgtccactgctttctaacaagataataaaccccccccctcttttctttttctttatttttatttcttttagctatttgatcctttctgaagcagttgtttctggaagagtctgtgcgcccatggatggctgagcaccactacgacttagtccgggataagggcctccccagtcctctccgggagatgatttgggaaattttataatgcttgttctgttaactcaccgggaccttgagggtccaatgggaccttgagggttttctctgaaatatacaaacttaaaggactctctctgaggttctttgactgacgtccactctcagtctggcccctgtgctcccctgtgtgtaccctggagtttctgtgtccaattgttggcatctaggtcttggctcaagattaggatgtgggccccactttagaggcacagactatgaaaagctgagttagtgcgcccgggacgccaggcaagcagcttttacagtttggcatcttattgcaggtgcttcgtgcacagtcagctgaaatagccaatgccaggtgctccaaccaccttatttccttgttttgttgattagaacaacacagaaaaaagcaaatataaatttttaatgactccatttaaaaatatcacagggtgggggcaaggaaattagctgagattcatctcaggattgagattctatccccccttccccgcccccagcagtgtcgctccaattcaaattagtggagaaaagattacagtaggccctgagccgactgtgaattcggtgcttggccaaggtaacactcatcgtattcacggagtgaaatactatatgatgatagttattatattatatgacgacttcattcacttcccaaatcacagggt

SEQ ID NO: 80 - DNA, 1695 bp, Homo sapiens
gcgggacggaagagggggtgaaggccagaggctcggggcttcaagaccgctgtctggagtccccctttccaggccatgtcggggcccacctggctgcccccgaagcagccggagcccgccagagcccctcaggggagggcgatcccccgcggcaccccggggccaccaccggcccacggagcagcactccagccccaccccagggtcaatttttgcccccttccatctgagcagtgttaccaggccccagggggaccggaggatcgggggccggcgtgggtggggtcccatggagtactccagcacacgcaggggctccctgcagacagggggggccttcgccctggaagcctggacgccgagatagacttgctgagcagcacgctggccgagctgaatgggggtcggggtcatgcgtcacggcgaccagaccgacaggcatatgagcccccgccacctcctgcctaccgcacgggctccctgaagccaaatccagcctcgccgctcccagcgtctccctatgggggccccactccagcctcttacactaccgccagcaccccggctggcccagccttccccgtgcaagtgaaggtggcacagccagtgaggggctgcggcccacccaggcggggagcctctcaggcctctgggcccctcccgggcccccactttcctctcccaggccgaggtgaagtctgggggcctggctataggagccagagagagccagggccaggggccaaagaggaagctgctggggtctctggccctgcaggaagaggaagaggaggcgagcacgggccccaggtgcccctgagccagcctccagaggatgagctggataggctgacgaagaagctggttcacgacatgaaccacccgcccagcggggagtactttggccagtgtggtggctgcggagaagatgtggttggggatggggctggggttgtggcctttgatcgcgtctttcacgtgggctgctttgtatgttctacatgccgggcccagcttcgcggccagcatttctacgccgtggagaggagggcatattgcgagggctgctacgtggccaccctggagaaatgtgccacgtgctcccagcccatcctggaccggatcctgcgggctatggggaaggcctaccaccctggctgcttcacctgcgtggtgtgtcaccgcggcctcgacggcatccccttcacagtggatgctacgagccagatccactgcattgaggactttcacaggaagtttgccccaagatgctcagtgtgcggtggggccataatgcctgagccaggtcaggaggagactgtgagaattgttgctctggatcgaagttttcacattggctgttacaagtgcgaggagtgtgggctgctgctctcctctgagggcgagtgtcagggctgctacccgctggatgggcacatcttgtgcaaggcctgcagcgcctggcgcatccaggagctctcagccaccgtcaccactgactgctgagtcttcctagaagtacctgctgggttctcagttccagttcccatcctttgattgatcactctccctgacatccacctgtatgactttgtcaccaaatgctgtcttctctttctccaatcaagaaataataatccctcgagtttacaaaacaaaaaaaaaaaaaaaaaaaaaaaaaaaaaaaaaaaaaaa

SEQ ID NO: 81 - DNA, 2301 bp, Homo sapiens
agcagagcggacgggcgcgcgggaggcgcgcagagctttcgggctgcaggcgctcgctgccgctggggaattgggctgtgggcgaggcggtccgggctggcctttatcgctcgctgggcccatcgtttgaaactttatcagcgagtcgccactcgtcgcaggaccgagcggggggcgggggcgcggcgaggcggcggccgtgacgaggcgctcccggagctgagcgcttctgctctgggcacgcatggcgcccgcacacggagtctgacctgatgcagacgcaagggggttaatatgaacgcccctctcggtggaatctggctctggctccctctgctcttgacctggctcacccccgaggtcaactcttcatggtggtacatgagagctacaggtggctcctccagggtgatgtgcgataatgtgccaggcctggtgagcagccagcggcagctgtgtcaccgacatccagatgtgatgcgtgccattagccagggcgtggccgagtggacagcagaatgccagcaccagttccgccagcaccgctggaattgcaacaccctggacagggatcacagcctttttggcagggtcctactccgaagtagtcgggaatctgcctttgtttatgccatctcctcagctggagttgtatttgccatcaccagggcctgtagccaaggagaagtaaaatcctgttcctgtgatccaaagaagatgggaagcgccaaggacagcaaaggcatttttgattggggtggctgcagtgataacattgactatgggatcaaatttgcccgcgcatttgtggatgcaaaggaaaggaaaggaaaggatgccagagccctgatgaatcttcacaacaacagagctggcaggaaggctgtaaagcggttcttgaaacaagagtgcaagtgccacggggtgagcggctcatgtactctcaggacatgctggctggccatggccgacttcaggaaaacgggcgattatctctggaggaagtacaatggggccatccaggtggtcatgaaccaggatggcacaggtttcactgtggctaacgagaggtttaagaagccaacgaaaaatgacctcgtgtattttgagaattctccagactactgtatcagggaccgagaggcaggctccctgggtacagcaggccgtgtgtgcaacctgacttcccggggcatggacagctgtgaagtcatgtgctgtgggagaggctacgacacctcccatgtcacccggatgaccaagtgtgggtgtaagttccactggtgctgcgccgtgcgctgtcaggactgcctggaagctctggatgtgcacacatgcaaggcccccaagaacgctgactggacaaccgctacatgaccccagcaggcgtcaccatccaccttcccttctacaaggactccattggatctgcaagaacactggacctttgggttctttctggggggatatttcctaaggcatgtggcctttatctcaacggaagccccctcttcctccctgggggccccaggatggggggccacacgctgcacctaaagcctaccctattctatccatctcctggtgttctgcagtcatctcccctcctggcgagttctctttggaaatagcatgacaggctgttcagccgggagggtggtgggcccagaccactgtctccacccaccttgacgtttcttctttctagagcagttggccaagcagaaaaaaaagtgtctcaaaggagctttctcaatgtcttcccacaaatggtcccaattaagaaattccatacttctctcagatggaacagtaaagaaagcagaatcaactgcccctgacttaactttaacttttgaaaagaccaagacttttgtctgtacaagtggttttacagctaccacccttagggtaattggtaattacctggagaagaatggctttcaatacccttttaagtttaaaatgtgtatttttcaaggcatttattgccatattaaaatctgatgtaacaaggtggggacgtgtgtcctttggtactatggtgtgttgtatctttgtaagagcaaaagcctcagaaagggattgctttgcattactgtccccttgatataaaaaatctttagggaatgagagttccttctcacttagaatctgaagggaattaaaaagaagatgaatggtctggcaatattctgtaactattgggtgaatatggtggaaaataatttagtggatggaatatcagaagtatatctgtacagatcaagaaaaaaaggaagaataaaattcctatatcat

(19) United States
(12) Patent Application Publication
(10) Pub. No.: US 2013/0150255 A1
(43) Pub. Date: Jun. 13, 2013

(54) DNA METHYLATION BIOMARKERS OF LUNG FUNCTION

(71) Applicants: Edward L. Murrelle, Midlothian, VA (US); Barbara K. Zedler, Richmond, VA (US); Andrew R. Joyce, Richmond, VA (US); Edwin J.C.G. van den Oord, Richmond, VA (US); Tapas K. Sengupta, Springfield, VA (US); Hailong Meng, New York, NY (US); Daniel E. Adkins, Richmond, VA (US)

(72) Inventors: Edward L. Murrelle, Midlothian, VA (US); Barbara K. Zedler, Richmond, VA (US); Andrew R. Joyce, Richmond, VA (US); Edwin J.C.G. van den Oord, Richmond, VA (US); Tapas K. Sengupta, Springfield, VA (US); Hailong Meng, New York, NY (US); Daniel E. Adkins, Richmond, VA (US)

(21) Appl. No.: 13/541,507

(22) Filed: Jul. 3, 2012

Related U.S. Application Data
(63) Continuation of application No. PCT/US11/20152, filed on Jan. 4, 2011.
(60) Provisional application No. 61/292,153, filed on Jan. 4, 2010.

(51) Int. Cl. C12Q 1/68

(52) U.S. Cl. CPC C12Q 1/6876
USPC 506/9; 506/16

(57) ABSTRACT

Biomarkers of lung disease are provided. The biomarkers comprise target genomic DNA sequences having one or more CpG dinucleotides that are differentially methylated in genomic DNA of subjects having lung disease as compared to normal subjects or subjects not having lung disease. In one exemplary embodiment, methylation status profiles of 71 CpG sites mapping to 67 unique genes are significantly associated with at least one of three lung function decline measures associated with lung disease. Other biomarkers significantly associated with cigarette smoking-related lung function decline, with age-related lung function decline, and with the intensifying effects of cigarette smoking on lung function decline with age are also provided.

This application claims the benefit of U.S. Provisional Application Ser. No. 61/292,153, filed Jan. 4, 2010, the entirety of which is hereby incorporated by reference.

### FIELD OF THE TECHNOLOGY

The field of the technology provided herein relates generally to pulmonary and related diseases and diagnosis and prognosis thereof.

### BACKGROUND

Pulmonary diseases impair lung function and, according to the American Lung Association, are the third primary cause of death in America; accounting for one in six deaths. The main categories of lung disease include airway diseases, lung tissue diseases and pulmonary circulation diseases, as well as combinations of the above. Examples of diseases affecting lung function include asthma, chronic obstructive pulmonary disease, influenza, pneumonia, tuberculosis, lung cancer, pulmonary fibrosis, sarcoidosis, HIV/AIDS-related lung disease, alpha-1 antitrypsin deficiency, respiratory distress syndrome, bronchopulmonary dysplasia and embolism, among others.

Chronic obstructive pulmonary disease (COPD) is the fourth leading cause of morbidity and mortality in the United States and is expected to rank third as the cause of death, worldwide, by 2020 (Rabe et al., Am J Respir Crit Care Med 2007, 176:532-555; Mannino et al., Proc Am Thorac Sac 2007, 4:502-506). The operational diagnosis of lung diseases such as COPD has traditionally been made by spirometry, as a ratio of the forced expiratory volume in one second (FEV₁) to the forced vital capacity (FVC) below 70% (Rabe et al., 2007). Cigarette smoking is recognized as the most important causative factor for COPD (Rabe et al., 2007; Mannino et al., 2007; Marsh et al., Eur Respir J 2006, 28:883-884). It is estimated that up to 50% of smokers may eventually develop COPD, as defined by spirometric guidelines of the Global Initiative for Chronic Obstructive Lung Disease (GOLD) (Mannino et al., 2007; Løkke et al., Thorax 2006, 61:935-939; Lundbäck B et al., Respir Med 2003, 97:115-122).

COPD is characterized by progressive, not completely reversible airflow limitation resulting from small airway disease (obstructive bronchiolitis) and alveolar and connective tissue destruction (emphysema) caused by chronic inflammation and structural changes from repeated injury and repair (Rabe et al., 2007). The underlying pathophysiological mechanisms identified in COPD include an imbalance between protease and anti-protease activity in the lung, oxidative stress with dysregulation of anti-oxidant activity, and chronic abnormal inflammatory response to long-term inhalation of toxic particles and gases (Rabe et al., 2007; Barnes PJ, Annu Rev Med 2003, 54:113-129; Barnes et al., Eur Respir J 2003, 22:672-688). In addition to local pulmonary inflammation, COPD is associated with significant systemic complications that may be due to a low-grade, chronic systemic inflammation (Agusti et al., European Respiratory Journal 21.2 (2003): 347-60; Agusti et al., Journal of Chronic Obstructive Pulmonary Disease 5 (2008): 133-38; Rahman et al., American Journal of Respiratory and Critical Care Medicine 154.4 Pt I (1996): 1055-60; Fabbri et al., Lancet, 370 (2007): 797-99). Although the airflow obstruction component of COPD has been traditionally assessed by spirometry, this tool does not adequately reflect, or predict, COPD's multidimensional, systemic involvement. Moreover, lung function tests, like spirometry, that provide a general assessment of lung function, do not distinguish between the different types of lung diseases that may be present (e.g., COPD, asthma, fibrosis, emphysema), and cannot be used to confirm a diagnosis alone. In addition, it is only when a change in lung function exists can such tests assist in the diagnosis of lung disease.

In light of the foregoing, biomarkers, or molecules that reflect the pathobiological disease process, may be useful for diagnosing or predicting clinical outcomes of COPD as well as for assessing new therapies that modify the underlying disease process (inflammation, oxidative stress, tissue destruction). Indeed, several cytokines, including leptin (Broekhuizen et al., Respir Med 2005, 99:70-74), tumor necrosis factor-alpha (TNF-α), interleukin 8 (IL-8) (Drost et al., Thorax 2005, 60:293-300) and Clara cell 16 protein (Braido et al., Respir Med 2007, 101:2119-2124) hold promise to be useful biomarkers of COPD. An ideal biomarker is directly indicative of the pathogenic process, easily measured, reproducible, and sensitive to effective intervention (Stockley R A. Thorax 2007, 62:657-660).

Unlike genetic modifications in the form of DNA mutations, epigenetic changes are potentially reversible, can happen in one's lifetime and therefore may be treatable or preventable through drugs, diet modification and/or supplementation, and other environmental interventions such as smoking cessation (Gallou-Kabani et al., Diabetes 2005, 54:1899-1906; Foley et al., Am J Epidemiol 2009, 169:389-400). Indeed, the importance of epigenetic abnormalities in diseases and their potentially reversible nature is underscored by the recent approval by the US Food and Drug Administration of three drugs (Vidaza®, Dacogen® and Zolinza™) that inhibit key enzymes responsible for epigenetic changes, such as DNA methyltransferases and histone deacetylases, for the treatment of acute myelogenous leukemia and myelodysplastic syndrome (Desmond et al., Leukemia 2007, 21:1026-1034; Yuan et al., Cancer Res 2006, 66:3443-3451).

### SUMMARY

DNA methylation plays an important role in determining whether some genes are expressed; thus it is an essential control mechanism for controlling the normal functioning of cells and organ systems in an individual. Aberrant DNA methylation (as compared to methylation status in normal healthy cells) is one mechanism underlying loss of expression of genes important for maintaining a healthy state in an individual. As epigenetic changes, such as DNA methylation, can precede symptomatic stages of many diseases, such changes, if detectable, serve as important biomarkers for early detection and prognosis (Tsou et al., Oncogene 2002, 21:5450-5461). Current studies of mechanisms underlying lung diseases are hampered by the invasive procedures required to obtain samples of disease tissue for study. In contrast to gene expression markers, which are RNA-based, some epigenetic markers, such as DNA methylation, employ DNA-based assays. Due to the higher stability of DNA as compared to RNA, analysis of DNA methylation as a marker of gene expression can be accomplished using biological samples that are otherwise non-informative when using RNA-based techniques. It is known that, in disease states, DNA methylation is not limited to the affected tissue or cell type, but can be detected in peripheral biofluids. Studies of gene regulation using methylation assays can be performed on any biological sample containing DNA including, for example, archived fixed tissue and biofluids obtained by minimally invasive procedures (e.g., aspirate, blood, sputum, etc.) (Robertson K D: Nat Rev Genet 2005, 6:597-610). These attributes make DNA methylation profiling a powerful tool for identifying diagnostic/prognostic biomarkers, as well as for understanding disease mechanisms (Robertson K D: Nat Rev Genet 2005, 6:597-610).

Lung function and its decline are affected by a number of biological and environmental factors, especially gender, age and cigarette smoking (Hoidal J R. Eur Respir J 2001, 18:741-743; Feenstra et al., Am J Respir Crit Care Med 2001, 164:590-596; Connett et al.: Design of the Lung Health Study: a randomized clinical trial of early intervention for chronic obstructive pulmonary disease, Control Clin Trials 1993, 14:3 S-19S). In the presence of such etiological complexity, conventional analytical strategies, such as using COPD/non-COPD disease status or reliance on simple spirometric measurements alone, are often inadequate. This disclosure assesses the association of these measures of lung function or decline with the DNA methylation profiles generated from the peripheral blood mononuclear cells (PBMCs) of 311 Lung Health Study (LHS) and Genetics of Addiction Project (GAP) participants with or without COPD using the high-throughput GoldenGate® DNA methylation platform (Illumina, La Jolla, Calif.).

As described herein, seventy-one CpG sites mapping to sixty seven unique genes are found to be significantly associated with at least one of three lung function decline measures associated with COPD (See Table 2). More specifically, as disclosed herein, forty five CpG sites are significantly associated with cigarette smoking-related lung function decline, thirty one CpG sites are significantly associated with age-related lung function decline, and one CpG site is significantly associated with the intensifying effects of cigarette smoking on lung function decline with age (CCR5, minimum overall p-value=8.63×40 10⁻⁵).

Novel biomarkers of lung function are provided. The compositions, methods and kits disclosed herein relate to the discovery of the association between lung disease and the methylation profile of a number of genes. In particular, the methylation states of certain dinucleotide sequences have significant novel associations with COPD. As described below, the methylation changes are located at certain CpG sites within genes involved in biological processes such as inflammation, inter-cellular signaling (endocrine system) and DNA damage repair. The genes and CpG sites associated with COPD described herein are listed in Tables 2 and 3.

In one embodiment, a method is provided for identifying one or more biomarkers of lung disease comprising comparing a DNA methylation profile obtained from a sample of lung disease tissue to a DNA methylation profile from a sample of normal or non-diseased tissue. Exemplary lung diseases include, for example, COPD, obstructive pulmonary disease, chronic systemic inflammation, emphysema, asthma, pulmonary fibrosis, cystic fibrosis, obstructive lung disease, pulmonary and inflammatory disorder. Thus, a biomarker of lung disease may be a CpG site, dinucleotide sequence and/or genomic target sequence having one or more CpG sites that are differentially methylated in a genomic DNA sample obtained from an individual having one phenotypic status (e.g. having a lung disease such as, for example, COPD) as compared with the methylation status of corresponding CpG site(s) in genomic DNA obtained from an individual having another phenotypic status (e.g. healthy subject not having lung disease). A biomarker is characterized by its association with a particular lung disease such as COPD. Exemplary analytical methods for determining statistical significance include Ordinary Least Squares (OLS) regression with different outcome variables. Outcome variables can include, for example, age, ethnic origin, sex, life style, patient history, drug response and others

In one aspect, characterization of a CpG site as a biomarker may also include use of an algorithm to identify those CpG sites having low or no inter-individual variability in methylation status for the disease outcome assessed. The non-variable sites are excluded from the subsequent association analysis thereby reducing false-positive findings and increasing the statistical power for identifying a CpG site as a biomarker of the selected disease. See the examples, including Example 2.

In another embodiment, a method is provided for diagnosing or aiding in the diagnosis of lung disease by (i) assessing the methylation profile of one or more gene(s), DNA region(s) and/or CpG site(s) in a sample of genomic DNA obtained from a subject suspected of having a lung disease and (ii) comparing the results to a reference methylation profile, wherein the reference profile includes a known standard DNA methylation biomarker. Assessing the methylation profile includes identifying the DNA methylation profile for two or more preselected target CpG sites, and comparing the results to a reference profile, wherein the reference profile includes a known standard biomarker (e.g. known DNA methylation profile associated with a lung disease such as COPD). In one embodiment, the method comprises assessing the methylation profile of highly variable CpG sites. In one embodiment, the biomarker is one or more CpG target site(s) selected from those provided in Tables 2 and 3.

In another embodiment, the present disclosure provides a method for determining a subject's relative risk of developing a lung disease comprising assessing the DNA methylation profile of one or more gene(s), DNA region(s) and/or CpG site(s) in a sample of genomic DNA obtained from a subject and comparing the results to a reference methylation profile wherein the reference profile is a DNA methylation profile associated with an increased risk of developing lung disease. In one embodiment, the method comprises assessing the methylation profile of highly variable CpG sites. In one aspect, the reference profile includes one or more target CpG site(s) selected from those provided in Tables 2 and 3.

In another embodiment, methods are provided for monitoring the course of progression, or managing the treatment, of a lung disease such as COPD in a subject comprising: (a) measuring at least one biomarker in a first biological sample from the subject, wherein the at least one biomarker specifically indicates the presence of a lung disease; (b) measuring the at least one biomarker in a second biological sample from the subject, wherein the second biological sample is obtained from the subject after the first biological sample; and (c) correlating the measurements with a progression or regression of lung disease in the subject. In one aspect, measuring at least one biomarker includes determining a DNA methylation profile for two or more preselected target CpG sites. In a particular embodiment, a preselected target CpG site is selected from those provided in Tables 2 and 3.

In one embodiment, determining a DNA methylation profile employs array or microarray technology, such as, for example, an array platform that allows for high-throughput sample handling and data processing. In one embodiment, an array or microarray permits methylated and non-methylated sites to be distinguished (e.g., by distinguishing between nucleic acid sequences that have been exposed to methylation sensitive restriction endonucleases).

In another embodiment, the present disclosure provides a kit which can be used, for example, in performing one or more of the methods described herein. The kit includes a composition comprising a positive control, a composition comprising a negative control, and a pamphlet describing use of the compositions in an assay for obtaining a DNA methylation profile. In one embodiment, the positive control includes DNA having a known DNA methylation profile associated with a lung disease such as COPD. In some embodiments, the positive control includes DNA having a CpG site selected from those provided in Tables 2 and 3. In other embodiments, the kit may also include a standard dataset of a DNA methylation profile associated with at least one phenotypic measure of lung function or with a preselected lung disease or impairment of lung function.

In another embodiment, the present disclosure provides biomarkers used for diagnosing, prognosing, management of treatment, or monitoring lung disease in a subject comprising one or more methylated CpG sites of nucleic acids in one or more genes selected from the group consisting of CCR5 gene and the genes listed in Table 2 or Table 3.

In another embodiment, the present disclosure provides the use of one or more, two or more, three or more, four or more, or five or more, methylated CpG sites of nucleic acids in one or more, two or more, three or more, four or more, or five or more, genes selected from the group consisting of CCR5 gene and the genes listed in Table 2 or Table 3 as a biomarker for diagnosing, prognosing, managing the of treatment of, or monitoring lung disease, in a subject.

### BRIEF DESCRIPTION OF THE DRAWINGS

FIG. 1 shows an interaction network and selected disease links for genes with methylated CpG sites that are significantly associated with the pack-years decline lung function measure. Genes associated with 18 of the CpG sites significantly associated with pack-years decline form a subnetwork in which each gene is linked to at least one other by way of direct binding or regulation. Each of these genes, as well as 11 other genes with methylation significantly associated with the pack-years decline measure, is linked to at least one disease or disease process associated with COPD (oxidative stress-related DNA damage, mutagenicity, inflammation) or associated pulmonary disorders (e.g., lung diseases such as lung cancer, lung disease, asthma, emphysema). The genes significantly associated with pack-years decline also include many linked to extracellular matrix remodeling or hematopoesis and several linked to the Wnt-signalling pathway.

FIG. 2 shows an interaction network and selected disease links for genes with methylated CpG sites that are significantly associated with the age-decline lung function measure. Genes associated with 9 of the CpG sites significantly associated with age-decline form a subnetwork in which each is linked to at least one other by way of positive or negative regulation. Each of these genes, as well as 7 additional genes with methylation significantly associated with the age-decline measure, are linked to at least one disease or disease process associated with COPD (oxidative stress-related DNA damage, mutagenicity, inflammation) or pulmonary disorders (e.g., lung diseases such as cancer, lung disease, asthma, emphysema). The genes significantly associated with age-decline also include many linked to inflammation either directly or through association with TGFβ signaling, many linked to the endocrine system, and two components of the retinoic acid pathway.

FIG. 3 is a graph of probe correlations versus total probe variance. The relationship between probe correlations and total probe variances is shown. Relatively high total probe variance corresponds to a high probe correlation across technical replicates, which suggests that low probe correlations are due to low variances between biosamples.

FIG. 4 shows a plot of the distribution of probe correlations. The distribution of probe-level correlations across technical replicates for each probe is shown. Pearson correlation coefficients were calculated for the 1,505 CpG probes using 126 replicate biosamples distributed across five methylation matrices. The mean of the probe correlations is 0.268. The apparent bi-modality of this distribution suggests that probes come from two different groups, one comprising biologically relevant probes that exhibit high correlations, and another with low methylation-associated variance that may be excluded from subsequent analyses.

FIG. 5 shows the posterior probability distribution from mixture model. The posterior probability distribution, indicating the likelihood of a probe belonging to the subset of highly correlated informative probes, is displayed in blue. The green line indicates the number of probes (y-axis) that will remain at different posterior probability thresholds (x-axis) calculated from the two-class mixture model.

FIG. 6 shows the results of a False Discovery Rate (FDR) analysis. Panel (A), shows a plot of the number of significant probes detected at different q-values (from the regression analyses between DNA methylation changes) prior to probe selection as described herein for four outcome measures of lung function or decline (i.e., Age Decline, Pack-Years Decline, CPDX Age Decline and Baseline Lung Function). Panel (B) shows the number of significant probes detected at different q-values after probe selection for the same measures of lung function or decline used in Panel A. A greater number of significant probes was identified for a given q-value cutoff for age-decline, CPD x age-decline and Baseline lung function outcomes after probe selection.

### DETAILED DESCRIPTION

The present disclosure relates to the discovery of novel epigenetic changes associated with lung disease. More specifically, as described herein, methylation of certain genomic dinucleotide sequences is associated with phenotypic measures of lung diseases and disorders such as Chronic Obstructive Pulmonary Disease (COPD) (after controlling for the effects of age and baseline lung function). Methylations of such dinucleotide sequences are useful as biomarkers of lung disease such as COPD. Thus, in various embodiments, the present disclosure is based, in part, on the identification of reliable biomarkers associated with lung disease and its clinical progression. Exemplary lung diseases include COPD, obstructive pulmonary disease, chronic systemic inflammation, emphysema, asthma, pulmonary fibrosis, cystic fibrosis, obstructive lung disease, and pulmonary inflammatory disorder.

Expression of epigenetic markers is not restricted to the affected tissue or cell type to which the disease marker is associated, and therefore aberrantly methylated CpG sites can be detected in DNA isolated from peripheral biofluids of diseased subjects. For example, with IGF2 (an epigenetic locus), methylation imprinting can be detected in lymphocytes as well as the colon, although that methylation marker is associated with an increased colorectal cancer risk (Rakyan et al., Biochem. J. 2001, 356:1-10). Thus, systemic epigenetic changes that predate the onset of disease can be present in peripheral blood cells (Bracke et al., Clin Exp Allergy 2007, 37:1467-1479).

Studies of peripheral blood-based cells also reveal that methylation changes may predate or result from the epigenetic reprogramming events arising in germ line cells or early embryogenesis (Rakyan et al., Biochem. J. 2001, 356:1-10; Yeivin et al., (2008) Gene methylation patterns and expression. In Jost, J. and Saluz, H. (eds), DNA methylation: molecular biology and biological significance. Birkhauser-Verlag, Basel, pp. 523-568; Efstratiadis, A. (1994) Curr. Opin. Genet. Dev., 4, 265-280; Monk, et al., (1987) Development, 99, 371-382). Because the epigenetic profile of somatic cells is mitotically inherited, these epigenetic mutations are found in cells from peripheral blood. Also, blood contains proteins, metabolites, cells that have been modified as they circulate through diseased tissues, as well as cell-free DNA from diseased tissues and cells. As such, traces of the aberrant methylation in diseased target tissue may be present in peripheral biofluids. However, because sampled peripheral biofluid may not directly represent the methylation status of the diseased tissue, the present disclosure also provides a method for filtering out non-variable CpG sites, thereby increasing the statistical power to detect informative CpG sites useful as disease biomarkers.

### DEFINITIONS

A gene as used herein includes the exons (e.g., protein coding regions), introns, promoter, and any regulatory regions (e.g. 5′ upstream and 3′ downstream sequence). In some embodiments, a regulatory region is defined as a region that extends from sequence encoding a transcribed RNA to a point on the same DNA strand (chromosome) that, when methylated, alters the expression of the transcribed RNA, without encompassing another sequence encoding a different RNA. Unless stated otherwise, a gene includes both the coding and the non-coding DNA strand.

Diagnosing as used herein is the identification of a disease, disorder or condition in a subject.

Prognose, prognosticate, provide a prognosis, or prognosing, as used herein means to describe the likely outcome of a disease. As used herein with regard to lung disease or pulmonary disease, prognosis includes the outcome of a rapid decline or a slow decline in lung function.

Predicting the likelihood of developing a lung disease or impaired lung function, as used herein, is meant to describe a possibility of an individual developing a lung disease or impaired lung function.

Recognition sequences as used herein are nucleotide sequences that permit the identification or isolation of a nucleic acid molecule and that are separate (located in a different portion of a nucleic acid molecule) from the sequence of a gene (e.g., a gene found in Table 2 or 3), or a portion of the sequence of a gene, that the nucleic acid molecule may contain. In some embodiments, a recognition sequence may be sequence(s) that can be used to bind nucleic acid molecules to a an array or to bind to a substrate (e.g., a recognition sequence that hybridizes with to nucleic acid molecule covalently bound to locations in a spatially addressable array or on the surface of a bead/particle).

Examining the methylation of a CpG site refers to determining the methylation state of any CpG site by chemical, physical (e.g., mass spectroscopic) or biochemical means, or examining the results of any physical, chemical, or biochemical analysis that were used to determine the methylation state of a CpG site.

Obtaining a methylation profile means examining the methylation of a nucleic acid sample of a subject at one or more CpG sites. In some embodiments, the sites may be one or more sites found in a nucleic acid sequence corresponding to a gene selected from those listed in Table 2 or Table 3.

A control sample, as used herein, is a biological sample (e.g., a sample of DNA or DNA containing cells) from a subject or population of subjects (employed singly, or as a pool) that is known to have or not have a lung disease or impaired lung function. In one embodiment, a control sample is a DNA sample comprising a known methylation profile or DNA methylation status that is associated with a healthy, non-diseased phenotypic status. Alternatively, in one embodiment, a control sample may be a biological sample from a subject or a population pool having a known diagnosis of a particular pulmonary/lung disease (e.g., COPD), or may be a DNA sample comprising a known DNA methylation profile or DNA methylation status that is associated with a particular lung disease such as COPD, or may be a sample including one or more genes, DNA regions, CpG sites, highly variable CpG sites, and/or informative dinucleotide sequences that are associated with a particular lung disease such as COPD. A control sample includes isolated nucleic acid sequences having known CpG sites associated with a phenotypic status such that, when the sample is assayed in parallel with another sample, methylation of the control CpG site(s) mimics methylation of the informative CpG sites in tissue of a subject having the phenotype (e.g. healthy, disease-free subject or subject diagnosed with a lung disease or impaired lung function).

A standard or standard sample, as used herein, is a sample from a subject who does not have a lung disease or impaired lung function, or a predisposition to develop a lung disease or impaired lung function. A standard is also a sample of isolated nucleic acid sequences having a known methylation profile associated with a lung disease or impaired lung function or risk of developing a lung disease or impaired lung function. Alternatively, a standard is a dataset or database of one or more CpG sites whose methylation status is associated with a lung disease or impaired lung function or a preselected functional measure of a lung disease or impaired lung function. In some embodiments, the dataset or database is obtained from the methylation profile derived from another standard. In some embodiments, the dataset or database includes a methylation profile derived from a control sample for all applicable comparisons. In other embodiments, a standard sample includes a control sample.

A lung disease or impaired lung function is a disease or disorder that affects the ability of a subject's pulmonary system to operate effectively or that causes a decline in a pulmonary function measure such as FEV₁. Pulmonary or lung diseases or disorders include, but are not limited to, airway diseases, lung tissue diseases and pulmonary circulation diseases as well as combinations of the above. Examples of diseases or disorders affecting lung function include asthma, chronic obstructive pulmonary disease (COPD), pulmonary inflammatory disorder, chronic systemic inflammation, asthma, pulmonary fibrosis, cystic fibrosis, obstructive lung disease, emphysema, sarcoidosis, alpha-1 antitrypsin deficiency, respiratory distress syndrome, bronchopulmonary dysplasia and embolism. Diseases or disorders affecting lung function may also include influenza, pneumonia, tuberculosis, and HIV/AIDS-related lung disease. For the purpose of this disclosure, any embodiment of pulmonary diseases or disorders may exclude cancers and/or tumors of the lung, airways, or of other respiratory tissues.

In one embodiment an individual or a population of individuals may be considered as not having lung disease or impaired lung function when they do not have clinically relevant signs or symptoms of lung disease. Thus, in various aspects, an individual or a population of individuals may be considered as not having chronic obstructive pulmonary disease, chronic systemic inflammation, emphysema, asthma, pulmonary fibrosis, cystic fibrosis, obstructive lung disease, pulmonary inflammatory disorder, or lung cancer when they do not manifest clinically relevant symptoms and/or measures of those disorders. In one embodiment, an individual or a population of individuals may be considered as not having lung disease or impaired lung function, such as COPD, when they have a FEV₁/FVC ratio greater than or equal to about 0.70 or 0.72 or 0.75. In another embodiment, an individual or population of individuals that may be considered as not having lung disease or impaired lung function are sex- and age-matched with test subjects (e.g., age matched to 5 or 10 year bands) current or former cigarette smokers, without apparent lung disease who have an FEV1/FVC ≧0.70 or ≧0.75. Individuals or populations of individuals without lung disease or impaired lung function may be employed to establish the normal pattern or measure of methylation at one or more methylation sites (e.g., CpG sites), or to provide samples (control or standard samples) against which to compare one or more samples (e.g., samples taken at one or more different first and second times) from a subject whose lung disease or lung function status may be unknown. In other embodiments, an individual or a population of individuals may be considered as having lung disease or impaired lung function when they do not meet the criteria of one or more of the above mentioned embodiments.

In one embodiment control subjects not having lung disease or impaired lung function, as used herein, are sex- and age-matched current or former cigarette smokers, without apparent lung disease who have FEV1/FVC ≧0.70. Age matching may be conducted in bands of several years, including 5, 10 or 15 year bands. Control subjects are preferably recruited from the same clinical settings. A control group is more than one, and preferably a statistically significant number of control subjects. Control subjects may be used as sources of control or standard samples.

Aspects of the present disclosure are directed to CpG site(s) in a nucleotide sequence and/or genomic sequence having one or more CpG site(s) that are differentially methylated in a genomic DNA sample obtained from an individual having one phenotypic status (e.g. having a lung disease such as, for example, COPD) as compared with the methylation status of corresponding CpG site(s) in a genomic DNA sample obtained from an individual (control or standard sample) having another phenotypic status (e.g. a subject not having lung disease). The CpG sites and the nucleotide sequences bearing them, that have differential methylation described herein below are biomarkers of lung disease or impaired lung function.

### Embodiments

1. Methods of Identifying Biomarkers of Lung Disease Based on DNA Methylation

Methods for identifying biomarkers of lung disease based upon the status of DNA methylation are provided. A biomarker is characterized by its association with a particular lung disease such as COPD.

For the purpose of this disclosure, a biomarker is differentially methylated between different phenotypic states if the level of methylation of the biomarker in individuals having different phenotypes is found to be different at a significant level. An exemplary statistical analysis includes Ordinary Least Squares (OLS) regression with different outcome variables. Outcome variables can include, for example, age, ethnic origin, sex, life style, patient history, drug response and others.

The present disclosure provides a method of identifying a DNA methylation biomarker by assessing one or more methylated CpG sites in biological samples obtained from subjects diagnosed as having a preselected lung disease, followed by statistical analysis to correlate specific CpG sites with the lung disease or a particular phenotypic measure of the lung disease. As noted above, exemplary statistical analysis includes OLS regression with different outcome variables including, but not limited to, age, ethnic origin, sex, life style, patient history, drug response and others. In one embodiment, the method comprises assessing the methylation status of highly variable CpG sites.

Methods are provided for the systematic identification, assessment, and validation of genomic targets having informative CpG sites (sites whose methylation can be associated with pulmonary function), and a systematic method for the identification and verification of the methylation of those CpG sites. Once identified and verified, such sites can be used alone or in combination with other CpG sites or data on the methylation of other CpG sites, for example, in a panel or array of biomarkers useful for diagnostic or prognostic assay of a lung disease.

In one embodiment, identification of a biomarker includes the use of methods disclosed herein to identify those CpG sites having low or no inter-individual variability in methylation status for the disease outcome assessed. The non-variable sites are excluded from the subsequent association analysis, thereby reducing false-positive findings and increasing the statistical power for identifying a CpG site as a biomarker of the selected disease. See Example 2.

2. Methods of Diagnosing, Prognosing or Predicting the Likelihood of Developing a Lung disease or Impaired Lung Function and Analysis of Tissues

2.1 Methods of Diagnosing, Prognosing or Predicting the Likelihood of Developing a Lung Disease or Impaired Lung Function

Biomarkers, alone or in combination, are useful as prognostic or diagnostic markers of lung disease; as markers of therapeutic effectiveness of a treatment for lung disease; as markers for determining an individual's relative risk of developing lung disease and/or as markers for managing the treatment of a lung disease in a subject. Such biomarkers are also useful in the methods disclosed herein as they enable detection of differentially methylated genomic CpG dinucleotide sequences associated with a lung disease, for example, COPD and asthma.

One or more biomarkers can be used to distinguish a lung disease condition from a healthy non-diseased condition or from a disease other than a lung disease. Diagnosis of lung disease, such as COPD, may include, but is not limited to, examination for the methylation status of 1 or more, 2 or more, 3 or more, 4 or more, 5 or more, 6 or more, 7 or more, 8 or more, 10 or more, 15 or more, 20 or more, or 30 or more preselected target CpG sites or dinucleotide sequences in a test sample obtained from a subject, wherein methylation of a target CpG site is indicative of or aids in the diagnosis of lung disease in the subject. A test sample is a biological sample obtained from a subject whose disease status is unknown or who is suspected of having a lung disease wherein the biological sample includes the subject's genomic DNA. In one embodiment, a target CpG site is selected from Table 2 and/or Table 3.

In another embodiment, a biomarker of lung disease includes one or more informative dinucleotide sequences and their corresponding genes or DNA regions. A dinucleotide sequence is considered “informative” if there is a statistically significant correlation between the methylation state of the sequence and a lung disease. For example, an informative dinucleotide sequence is a highly variable CpG site that is associated with a phenotypic measure of COPD when the CpG site is methylated. In one aspect, analysis for statistical significance includes preexclusion of those dinucleotide sequences that have low to no inter-individual variability for the particular disease outcome measure. In a particular embodiment, a biomarker gene or DNA region has an informative dinucleotide sequence comprising a CpG site selected from those listed in Table 2 and Table 3.

One aspect of the present disclosure provides methods for diagnosing a lung disease, such as COPD, or for aiding in the diagnosis of a lung disease. Such method(s) comprise obtaining a methylation profile of genomic DNA from a biological sample obtained from a subject (“test” sample), and comparing the profile to a standard sample. A “control” sample may be a DNA sample obtained from an individual or a population pool having a known diagnosis of a particular pulmonary/lung disease (e.g., COPD), or may be a sample comprising a group of nucleic acid sequences or dinucleotide sequences having a known DNA methylation profile associated with a particular lung disease such as COPD. In such a comparison, the methylation status of two or more preselected CpG sites (“target CpG site”) in the test sample, that is the same or similar to the methylation status of the same gene, DNA region, CpG sites and/or informative dinucleotide sequences in the standard, identifies the subject as having the lung disease or aids in the identification of the subject as having a lung disease such as COPD. In one embodiment, a target CpG site is selected from those listed in Tables 2 and 3. Obtaining a methylation profile may include assessing the methylation status of two or more target CpG sites of DNA from a subject suspected of having a lung disease, and comparing the results to a standard profile, wherein the standard profile is a dataset or database of known biomarkers associated with a selected lung disease or a select phenotypic measure of lung disease.

In one embodiment, the present disclosure provides a method of determining a subject's relative risk of developing a lung disease. Such a method comprises assessing the DNA methylation profile in a genomic DNA sample obtained from a subject and comparing the profile to a standard or a control sample. One specific lung disease is COPD. In one embodiment, a target CpG site is selected from those listed in Tables 2 and 3.

In another embodiment, the present disclosure provides a method for monitoring the course of progression of a lung disease in a subject comprising: (a) determining a DNA methylation profile of a genomic DNA sample obtained from a subject at a first time point; (b) determining a DNA methylation profile of a genomic DNA sample obtained from the subject at a second time point, wherein the second genomic DNA sample is obtained from the subject after the first genomic DNA sample; and (c) correlating a difference between the profile of the first sample and the profile of the second sample with a progression or regression of lung disease in the subject. In a particular embodiment, the DNA methylation profiles include assessment of the methylation status of at least one CpG site selected from those listed in Table 2 and Table 3.

Tables 2 and 3 also provide a population of gene targets having informative CpG sites whose methylation status is significantly associated with one or more phenotypic measures of lung disease. Such gene targets may be used in the methods provided herein. For example, a methylation profile of a test sample (genomic DNA sample from a subject whose disease state is unknown) may be determined by measuring the methylation status of two or more gene targets wherein each target has at least one informative CpG site. The methylation profile of the test sample may then be compared to a standard profile that is associated with a preselected phenotypic measure of lung disease to diagnose, aid in the diagnosis of, and/or determine the subject's risk of developing a lung disease. Exemplary gene targets having at least one informative CpG site are set forth in Table 2 and Table 3.

In one embodiment, the present disclosure provides a method for diagnosing or prognosing a lung disease or impaired lung function, or predicting the likelihood of developing a lung disease or impaired lung function, comprising examining the methylation of CpG sites within one or more genes selected from those listed in Table 2 or Table 3. In some embodiments, the one or more genes are 2 or more, 3 or more, 5 or more, 6 or more, 8 or more, 10 or more, 12 or more, 15 or more, 20 or more, 25 or more, or 30 or more genes recited in Table 2 or Table 3. In other embodiments, the one or more genes are associated with pack-year decline in lung function or with age-decline in lung function. In one embodiment, the genes associated with pack-year decline and age-decline are selected from: ACVR1C; ATP10A; HTR1B; KIAA; SOX1; and TRIP6 (see SEQ ID NOs: 71, 71, 74, 75, 79, and 80). In one embodiment, the methylation sites of those genes associated with pack-year decline and age-decline are selected from: ACVR1C_P363_F; ATP10A_P147_F; HTR1B_P222_F; KIAA1804_P689_R; SOX1_P294_F; and TRIP6_P1274_R.

In one embodiment, the present disclosure provides a method of managing a subject's lung disease whereby a therapeutic treatment plan is customized or adjusted based on the status of the disease. Exemplary therapeutic treatments for lung disease include, but are not limited to, administering to the subject one or more immunosuppressants, corticosteroids (e.g. betamethasone delivered by inhaler), Beta (β)-2-adrenergic receptor agonists (e.g., short acting agonists such as albuterol), anticholinergics (e.g., ipratropium, or a salt thereof delivered by nebuliser), and/or oxygen. In addition, where the lung disease is caused by or exacerbated by bacterial or viral infections, one or more antibiotics or antiviral agents may also be administered to the subject.

The status of a subject's lung disease may be determined by assessing the DNA methylation profile of the subject's genomic DNA and comparing that methylation profile to a methylation profile obtained from one or more subjects who have been diagnosed with a particular lung disease or impairment of lung function of a predetermined severity. As used herein, the term “status” refers to the degree of severity of a subject's lung disease or impairment of lung function such as, for example, the number, or degree of severity of symptoms presented or exhibited by the subject suffering from the lung disease. The symptoms associated with different forms of lung disease may differ between forms of lung disease or may overlap. For example, exemplary symptoms commonly associated with COPD include long-term swelling in the lungs, destruction or decreased function of the air sacs in the lungs, a cough producing mucus that may be streaked with blood, fatigue, frequent respiratory infections, headaches, dyspnea, swelling of extremities, and wheezing. A subject suffering from COPD may have from a few to all of these symptoms. A subject suffering from an early stage of COPD can exhibit one to two or a few symptoms.

Biological sources of genomic DNA sample include, but are not limited to, cells or cellular components which contain DNA, cell lines, biopsies, blood, esophageal lavage fluid, sputum, buccal mucosa, stool, urine, cerebrospinal fluid, ejaculate, and tissue embedded in paraffin. A sample may also be derived from a population of cells or from a tissue afflicted with a lung disease (e.g., a lung biopsy). The methylation pattern of a genomic DNA sample should be representative of the cell or tissue type of interest. Samples can be analyzed individually or as a pool, depending upon the purpose of the analysis. Exclusion of non-variable CpG sites is preferred when the source of genomic DNA sample is derived from peripheral biofluid. Methylation markers that can be measured in peripheral biofluids are favored for diagnostic and prognostic purposes because of the simple, non-invasive manner in which the biosamples can be collected while still being representative of the .subject's disease status.

2.2 Determination of Nucleic Acid Methylation

The methods provided herein may employ, as required, highly sensitive and accurate techniques for assessing or determining a DNA methylation profile. In one embodiment, a DNA methylation profile or methylation status of specific CpG sites within a gene or DNA region can be detected using array technology and methods employing arrays such as, for example, a nucleic acid microarray or a biochip bearing an array of nucleic acids. An array or biochip generally comprises a solid substrate having a generally planar surface to which a capture reagent (e.g., dinucleotide sequence-specific probe) is attached. For example, a plurality of different probe molecules can be attached to a substrate or otherwise be spatially distinguished in an array. A probe may be one or more nucleic acid sequences which anneal to a complementary nucleic acid sequence depending upon the methylation status of a CpG site within the complementary nucleic acid sequence. In one particular embodiment, each probe has a unique position on the array and is stably associated with the array. Exemplary arrays include slide arrays, silicon wafer arrays, liquid arrays, bead-based arrays, and miniaturized array platforms. A DNA methylation profile or methylation status of one or more CpG sites within a genomic target can also be identified using high-throughput or multiplexing and scalable automation for sample handling.

In another embodiment the arrays will permit the detection and/or quantitation of two, three, four, five, six, seven, eight, ten, fifteen or more different informative CpG sites associated with a lung disease such as, for example, COPD.

In other embodiments, a DNA methylation profile or methylation status of one or more informative CpG sites within a target gene can be determined using other methods known in the art. Exemplary methods include use of bisulfite treatment in conjunction with methylation-specific PCR employing primer sets that allow discrimination between methylated and unmethylated genomic DNA, combined bisulfite restriction analysis (COBRA) and/or DNA arrays and/or employment of a restriction enzyme-based technology which uses methylation sensitive restriction endonucleases for differentiation between methylated and unmethylated cytosines. Restriction enzyme based methods include, for example, restriction endonuclease digestion with methylation-sensitive restriction enzymes, which can be followed by Southern blot analysis or PCR. Restriction enzyme based methods also include restriction landmark genomic scanning (RLGS) and differential methylation hybridization (DMH). In methods employing methylation-sensitive restriction enzymes, the digested DNA fragments can be separated, for example, by gel electrophoresis and the methylation status of the sequence deduced by the particular fragments presented. A post-digest PCR amplification step may also be included wherein a set of oligonucleotide primers, one on each side of the methylation sensitive restriction site, is used to amplify the digested DNA. PCR products are not detectable where digestion of the methylation sensitive CpG site occurs. A DNA methylation profile or methylation status of one or more CpG sites can also be determined using mass spectrometric analysis, liquid chromatography-tandem mass spectrometry, gas-liquid chromatography and mass spectrometry. Examples of additional methods known in the art are described in Huang et al., Human Mol. Genet. 8, 459-70, 1999; Plass et al., Genomics 58: 254-62, 1999; Gonzalgo et al., Cancer Res. 57:594-599, 1997; and Toyota et al., Cancer Res. 59:2307-2312, 1999), each of which are hereby incorporated by reference in their entireties.

3. Compositions for use in Methods of Diagnosing, Prognosing or Predicting the Likelihood of Developing a Lung Disease or Impaired Lung Function

The materials and reagents for diagnosing a lung disease, for determining the prognosis of a lung disease or for use in the treatment or management of lung disease in a subject may be assembled together in a kit. A kit comprises one or more probes of methylation status and a control nucleic acid sequence where the control nucleic acid sequence includes a dinucleotide sequence that is known to be methylated in a preselected lung disease. In some embodiments, the kit includes a composition comprising a positive control, a composition comprising a negative control, and a pamphlet describing use of the compositions in an assay for obtaining a DNA methylation profile. In one embodiment, the positive control includes an isolated DNA having a known DNA methylation profile associated with a lung disease such as COPD. In some embodiments, the positive control includes an isolated nucleic acid sequence having one or more CpG sites selected from those provided in Tables 2 and 3.

In another embodiment, the present disclosure provides a composition which can be used as a standard or reference sample in a method described herein. The composition comprises a population of isolated genomic DNA having one or more gene targets where each target includes at least one informative CpG site as provided in Tables 2 and 3. Alternatively, the composition comprises a population of dinucleotide sequences having an informative CpG site as provided in Tables 2 and 3. Detection of the methylation status of the informative CpG sites provides a standard or reference DNA methylation profile depending upon user objective.

The present disclosure also provides compositions comprising two or more nucleic acid molecules; with each of said two or more nucleic acid molecules comprising a first nucleic acid sequence and an optional second nucleic acid sequence; wherein said first nucleic acid sequence in each of said two or more nucleic acid molecules comprises a nucleic acid sequence having at least 20 contiguous nucleotides (e.g., 20 nucleotides having at least one CpG site of interest) of a gene found in Table 2 or Table 3. In some embodiments of such compositions, the two or more nucleic acid molecules are 3 or more, 4 or more, 5 or more, 6 or more, 8 or more, 10 or more, 12 or more, 15 or more, 20 or more, 25 or more, or 30 or more nucleic acid molecules. In other embodiments, the two or more nucleic acid molecules each comprise a first nucleic acid sequence having at least 20 contiguous nucleotides of different genes found in Table 2 or Table 3.

In an embodiment, the two or more nucleic acid molecules of the compositions are 3 or more, 4 or more, 5 or more, 6 or more, 8 or more, 10 or more, 12 or more, 16 or more, 20 or more, 24 or more, or 30 or more nucleic acid molecules, wherein each of said 3 or more, 4 or more, 5 or more, 6 or more, 8 or more, 10 or more, 12 or more, 16 or more, 20 or more, 24 or more, or 30 or more nucleic acid molecules that each comprise a first nucleic acid sequence having at least 20 contiguous nucleotides (e.g., 20 nucleotides having at least one CpG site of interest) of different genes found in Table 2 or Table 3.

In another embodiment, the two or more nucleic acid molecules of the composition described herein may each comprise a first nucleic acid sequence having at least 20 contiguous nucleotides (e.g., 20 nucleotides having at least one CpG site of interest) of different genes found in Table 2 or Table 3. In some embodiments the compositions comprising two or more nucleic acid molecules comprise one or more nucleic acid molecule pairs, wherein each nucleic molecule acid pair comprises the same first nucleic acid sequence having at least 20 contiguous nucleotides of a different gene selected from the genes in Tables 2 or Table 3 or the CCR5 gene, and wherein the first nucleic acid sequence of said pair of nucleic acid molecules differ in their methylation at CpG sites.

In one embodiment, the composition may comprise a group of nucleic acids (3 or more, 4 or more, 6 or more, 8 or more, 10 or more, 12 or more, 14 or more, 16, or more, 20 or more, 24 or more, or 30 or more) each having a first portion of a nucleic sequence which differs in its methylation of at least one CpG site from a second portion of the same molecule. Thus, the disclosure encompasses compositions having the same sequence present with different methylation present on at least one CpG site, which may be viewed as pairs of methylated and unmethylated sequences. Compositions comprising one or more of such nucleic acid molecule pairs having nucleotide sequence with different methylation patterns may comprise 2 or more, 4 or more, 6 or more, 8 or more, 10 or more, 12 or more, 14 or more, 16, or more, 20 or more, 24 or more, or 30 or more different nucleic acid molecule pairs, wherein each of said pairs comprises a first nucleic acid sequence from a different gene found in Table 2 or Table 3.

In some embodiments, the compositions as disclosed above comprise at least one nucleic acid molecule having a dinucleotide sequence whose methylation status is associated with a lung disease or impaired lung function, or a phenotypic measure of a lung disease or impaired lung function.

The length of the portion of the first nucleic acid that is derived from the genes in Table 2 or Table 3 may be greater than about 20 contiguous nucleotides of sequence from those genes, and may be at least 22, 24, 26, 28, 30, 32, 35, 40, 50, 75, 100, or 200 contiguous nucleotides. Similarly, the length of the first nucleic acid segments from the genes in Table 2 or Table 3, will by necessity be less than or equal to the length of the gene, or alternatively, less than 250, 300, 350, 400, 450 or 500 nucleotides.

The compositions include an array wherein the nucleic acid molecules are arranged in a spatially addressable array format. In one embodiment, arrays have a spatially addressable format that comprises two or more locations each having at least one type of nucleic acid present. In an embodiment, nucleic acid molecules are covalently attached to the locations. In another embodiment, nucleic acid molecules are non-covalently attached to the locations. Nucleic acid molecules comprising a first nucleic acid sequence selected from the genes found in Table 2 or Table 3 may be attached to the locations in the array by hybridization to nucleic acid molecules covalently attached to the locations. Hybridization may be accomplished by a second nucleic acid sequence complementary to the nucleic acids covalently linked to the substrate on which the array is formed.

In further embodiments, the compositions as described above include one or more, two or more, three or more, four or more, five or more, or six or more different nucleic acid molecule(s) that have been treated with bisulfite (e.g., nucleic acid molecules with a first sequence from different genes listed in Tables 2 and/or 3).

Also provided for herein are kits that comprise the compositions described herein (e.g., compositions comprising two or more nucleic acids, arrays, etc.) and instructions for their use in diagnosing, prognosing, or predicting the likelihood of developing a lung disease or impaired lung function.

In addition to the methods described above, methods also are provided for diagnosing or prognosing a lung disease or impaired lung function, or for predicting the likelihood of developing a lung disease or impaired lung function, comprising examining the methylation of one or more CpG sites of one or more different first nucleic acid sequences in the compositions described herein. In one embodiment, the method employs one or more, two or more, three or more, four or more, six or more, eight or more, ten or more, twelve or more, sixteen or more or 30 or more different first nucleic acid sequences. In such embodiments, an increase in methylation of CpG sites in one or more of said nucleic acid molecules in a subject is indicative of an increased probability of developing a lung disease or impaired lung function, having a lung disease or impaired lung function, or suffering from a decline in pulmonary function as defined by the ratio of FEV₁ to FVC.

Other substitutions, modifications, changes and omissions may be made in the design, operating conditions and arrangement of the aspects and embodiments described herein without departing from the spirit of this disclosure. Additional advantages, features and modifications will readily occur to those skilled in the art. Therefore, this disclosure, in its broader aspects, is not limited to the specific details, and representative devices, shown and described herein. Accordingly, various modifications may be made without departing from the spirit or scope of the general inventive concept as defined, inter alia, by the appended claims and their equivalents.

All of the references cited herein, including patents, patent applications, and publications, are hereby incorporated in their entireties by reference.

### EXAMPLES

### Example 1

### DNA Methylation of Biomarkers of Lung Function

Association of lung function or decline measures with the DNA methylation profiles are generated from the peripheral blood mononuclear cells (PBMCs) of 311 Lung Health Study (LHS) and Genetics of Addiction Project (GAP) participants with or without COPD using the high-throughput GoldenGate® DNA methylation platform (Illumina, La Jolla, Calif.). The intention is to identify genes with differentially methylated CpG sites associated with lung function or its decline in smokers with or without COPD. The goals are: 1) to increase mechanistic understanding of individual differences in smoking-related lung function decline, and 2) to identify biomarkers predictive or reflective of smoking-associated COPD.

### Subjects.

Subjects were selected from participants in the Lung Health Study (LHS and Genetics of Addiction Project (GAP at the University of Utah study center. LHS was a prospective, randomized, multicenter clinical study sponsored by the National Heart, Lung, and Blood Institute which enrolled during 1986-1989 male and female cigarette smokers, aged 35-60 years, with mild or moderate COPD by lung spirometry (ratio of FENT₁ to forced vital capacity (FVC)<0.70 and FEV₁ 55% to 90% of predicted) but otherwise healthy (Meng et al. 2010. BMC Bioinformatics 11:227). Lung spirometry was performed and smoking status was assessed annually for 5 years. In the follow-on GAP study during 2003-2004, spirometry was again performed, smoking status assessed and blood samples for high throughput epigenetic analysis obtained from 145 subjects with COPD. For comparison, 76 adult cigarette smokers without COPD and 90 healthy never-smokers were also studied in GAP. Characteristics of the study groups are shown in Table 1. At the GAP assessment, 91/145 (63%) of the smokers with COPD and 33/76 (43%) of the smokers without COPD had quit smoking.

TABLE 1
Demographic, smoking history and lung function characteristics of the subjects.

| | | Subjects Without COPD (n = 166) | | |
|---|---|---|---|---|
| | Subjects with | Smokers | Never-Smokers | |
| Characteristic | COPD (n = 145)¹ | (n = 76)¹ | (n = 90) | p-value² |
| Male, n (%) | 97 (67) | 36 (47) | 38 (42) | <0.001 |
| Age, mean (SD) | 64.6 (6.3) | 58.8 (7.0) | 55.8 (7.7) | <0.001 |
| BMI (kg/m²), mean (SD) | 27.9 (5.0) | 29.6 (6.7) | 29.6 (6.7) | 0.045 |
| Cigarettes per Day³, | 20.3 (12.5) | 18.9 (11.5) | n/a | 0.42 |
| mean (SD) | | | | |
| Years Smoked, mean (SD) | 42.7 (9.4) | 35.8 (8.9) | n/a | <0.001 |
| Pack-Years⁴, mean (SD) | 55.5 (32.4) | 46.3 (27.7) | n/a | 0.036 |
| FEV₁ (L), mean (SD) | 2.2 (0.6) | 3.0 (0.7) | 3.2 (0.9) | <0.001 |
| FEV₁ % predicted, mean | 69.7 (17.1) | 101.3 (14.1) | 102.1 (17.1) | <0.001 |
| (SD) | | | | |
| FEV₁/FVC, mean (SD) | 55.5 (11.6) | 75.9 (5.7) | 77.1 (8.2) | <0.001 |

COPD, chronic obstructive pulmonary disease; BMI, bodymass index; FEV₁, forced expiratory volume in 1 s; FVC, forced ventilatory capacity; n/a, not applicable
¹91/145 (63%) of the cigarette smokers with COPD and 33/76 (43%) of the smokers without COPD had quit smoking (percentages based on non-missing responses).
²The Chi-square test was used to compare gender among groups. Student's t-test was used to compare COPD and non-COPD smoker groups with respect to Cigarettes per Day, Years Smoked, and Pack-Years. One-way ANOVA tests were used to compare the remaining variables across the three groups. In all cases except for BMI, Holm-Sidak post tests revealed significant differences between COPD participants and non-COPD participants, but not between non-COPD smokers and never-smokers.
³Current daily cigarette consumption of continuing smokers.
⁴Pack-Years = (average cigarettes smoked per day/20) × (years of smoking).

### Biosamples and Illumina GoldenGate® Methylation Assay.

A whole blood sample is collected by venipuncture from each subject in a sodium citrated EDTA Vacutainer tube and shipped on dry ice. The PBMCs are isolated (Puregene Kit, Gentra Systems, Inc, Minneapolis, Minn.), and DNA is extracted using the AllPrep DNA/RNA Mini Kit (Qiagen Inc., Valencia, Calif.) and stored at −70° C. The isolated DNA is analyzed using the GoldenGate® Methylation Cancer Panel I assay (Illumina, San Diego, Calif.) to assess the DNA methylation status of 1505 CpG sites from over 800 genes. A listing of the methylation sites present in that panel is publicly available from a variety of sources and may be found, for example, on line at the web site of the European Bioinformatics Institute at the following URL (www.ebi.ac.uk/microarray-as/aer/lob?name=adss&id=2485795087). In addition to providing the GoldenGate® Reporter Name (CpG methylation site name), the United States National Center for Biotechnology Information (NCBI, U.S. National Library of Medicine, 800 Rockville Pike, Bethesda, Md., 20894 USA) accession number and version is provided for each sequence (e.g., gene sequence or cDNA) in which a methylation site is identified. The NCBI accession/version numbers uniquely identify nucleic acid and/or protein sequences present in the NCBI database and are publicly available, for example, on the word wide web at www.ncbi.nlm.nih.gov. Where an NCBI accession number is provided for a nucleic acid sequence encoding a protein produced by a gene indicated herein (e.g., a cDNA sequence) the corresponding gene sequence is also available in the NCBI database.

Prior to methylation profiling, bisulfite conversion of the DNA samples is conducted using the EZ DNA Methylation Kit (Zymo Research Corp., Orange, Calif.) in a 96-well format, per manufacturer's protocol using 2 μg of genomic DNA. Following conversion, 250 ng of DNA is used for the GoldenGate® methylation assay. The BeadStudio Methylation Module is used to read fluorescent signals from scanned images collected from the Illumina Beadarray Reader.

### Methylation Data Processing.

The 311 DNA biosamples are analyzed using five Illumina GoldenGate® matrices. Technical replicates are obtained for 126 biosamples by analyzing each on two separate matrices. The methylation status, or so-called Illumina β-value, of each CpG site is calculated based on fluorescent intensities corresponding to the methylated allele (Cy5) and the unmethylated allele (Cy3). Prior to calculating β-values, however, measurement artifacts are removed by independently correcting Cy5 and Cy3 fluorescent intensities for background signal as well as differential bisulfite conversion levels between biosamples (described in detail in the Supplemental Materials of (Storey J. The Annals of Statistics 2003, 31:2013-2035). Following signal correction, the β-value methylation measurement y (denoted as such to distinguish it from the quantity calculated using the standard Illumina technique) for biosample i and CpG site j is calculated as the ratio of corrected fluorescent intensities from the methylated allele (Cy5) to the total corrected fluorescent signal from both the methylated allele (Cy5) and the unmethylated allele (Cy3) such that:

yᵢⱼ=Cy5ᵢⱼ/Cy5ᵢⱼ+Cy3ᵢⱼ

### Methylation CpG Site Probe Selection.

A method to estimate the proportion of CpG sites included on the GoldenGate® matrices that showed little inter-individual variation in the biosamples examined has been described by Storey et al. (The Annals of Statistics 2003, 31:2013-2035). Using that method invariant CpG sites are removed from subsequent analyses given that measurements at these sites reflect technical procedural errors, for example, in sample preparation or image processing, rather than true biological differences among the individuals. By removing invariant sites, the statistical power to detect significant associations with phenotype is increased and the potential of false positive results is reduced.

Using mixture modeling to estimate the posterior probabilities that CpG sites showed substantial variation in true methylation status or, alternatively, showed little variation in methylation status across biosamples, the correlations of CpG site methylation status across 126 biosamples is conducted. CpG sites showing little variation in methylation were discarded, and only the CpG sites exhibiting true biological variation across biosamples (posterior probability ≧0.5) are retained for subsequent tests of association with the lung function measures.

### Lung Function Measures.

Four measures of lung function or lung function decline, measured spirometrically as FEV₁ (Knudson, R. J. et al. (1983) Am. Rev. Respir. Dis., 127, 725-734), are derived from statistical modeling of lung function decline in COPD using the longitudinal LHS and GAP spirometric, smoking history, and demographic data employing linear mixed models (see Example 3). Conceptually, these measures represent different underlying biological processes driving lung function decline. For association testing the analysis is focused on age-related decline (age-decline), pack-years-related decline (pack-years decline), the intensifying effects of smoking, in terms of number of cigarettes per day (CPD) and decline with age (CPD x age-decline) that together accounted for the vast majority of individual differences in lung function decline in these subjects. Also included in the association testing is baseline lung function, measured at the subjects' entry into the study, as an outcome measure, as it has also been shown to vary in magnitude across individuals (Griffith, K. A. et al. (2001) Am. J. Respir. Crit. Care Med., 163, 61-68).

### Association Testing

Ordinary least squares regression analyses are used to test for association between CpG site DNA methylation status and lung function or decline measures. A separate regression is estimated for each of the selected CpG sites (predictor variable) with respect to each of the four lung function or decline measures (outcome variables). The F test statistic is used to perform significance tests. To control the risk of false discovery, a “q-value” for each association test is calculated. A q-value is an estimate of the proportion of false discoveries, or false discovery rate (FDR), among all significant markers when the corresponding p-value is used as the threshold for declaring significance (Storey et al., Proc Natl Acad Sci USA 2003, 100:9440-9445; Fernando et al., Genetics 2004, 166:611-619). This FDR-based approach (1) provides a good balance between the competing goals of true positive findings versus false discoveries, (2) allows the use of more similar standards in terms of the proportion of false discoveries produced across studies because it is much less dependent on an arbitrary number or set of statistical tests that are performed, (3) is relatively robust against the effects of correlated tests (Storey et al., Proc Nati Acad Sci USA 2003, 100:9440-9445; Benjamini Y et al., J. R. Stat. Soc. Ser. B 1995, 57:289-300; van den Oord EJCG: Mol Psychiatry 2005, 10:230-231; Zhang H. J Cell Physiol 2007, 210:567-574), and (4) provides a more subtle picture about the possible relevance of the tested markers rather than an all-or-nothing conclusion about whether a study produces significant results (Storey et al., Proc Natl Acad Sci USA 2003, 100:9440-9445; Benjamini Y et al., J. R. Stat. Soc. Ser. B 1995, 57:289-300; van den Oord EJCG: Mol Psychiatry 2005, 10:230-231; Zhang H. J Cell Physiol 2007, 210:567-574). The q-values are calculated conservatively assuming p0=1.

### Pathway Analysis and Visualization.

The Pathway Studio software package (Ariadne Genomics, Rockville, Md.) is used to identify and visualize molecular interactions between the loci significantly associated with the lung function or decline measures. The Pathway Studio ResNet database is also queried to identify links to selected pathobiological mechanisms commonly associated with COPD, such as oxidative stress (DNA damage and mutagenicity) and inflammation, as well as the pulmonary disorders asthma, lung disease, and lung cancer.

### Probe Selection to Eliminate Non-Informative Loci.

Using the described probe selection technique of Storey J et al (The Annals of Statistics 2003, 31:2013-2035), 634 of the 1505 CpG sites included on the Golden Gate Methylation Cancer Panel I for subsequent association testing with the lung function measures are retained. The selected CpG sites exhibited relatively high methylation variation across individuals (posterior probability 0.5) while maintaining high correlation across technical replicates. The statistical advantages of this probe selection technique are revealed by comparing association testing with all 1505 CpG sites relative to the selected subset. Across a range of statistical cutoffs, the number of significantly associated CpG sites is higher in the selected subset (Storey J: The Annals of Statistics 2003, 31:2013-2035), indicating improved statistical power as a result of using the described probe selection strategy.

Invariant probes of COPD might also be due to the use of a CpG panel that is originally designed primarily to study cancer-related methylation changes. Accordingly, the majority of CpG sites found on the array correspond to oncogenes and tumor suppressor genes. A smaller fraction of probes are associated with X-linked and known imprinted genes, as well as previously reported differentially methylated loci. However, COPD shares common pathobiological mechanisms with cancer, notably elevated oxidative stress and chronic systemic inflammation (Lin and Karin, J Clin Invest 2007, 117:1175-1183; Barnes P J. Proc Am Thorac Soc 2008, 5:857-864; Jin et al., Cytokine 2008, 44:1-8), and accordingly shares common genetic links and molecular pathways (Mohr et al., Trends Mol Med 2007, 13:422-432). As such, while designed primarily for cancer research, the GoldenGate® Methylation Cancer Panel I represent a useful tool for epigenetic examination of COPD.

Another contributor to invariant CpG probes found herein might be the use of DNA extracted from PBMCs rather than specific lung tissue or biofluids. In recent years, increasing evidence has shown that peripheral blood mononuclear cells can be used as a readily available and accessible target tissue ‘surrogate’ that accurately reflects disease or risk of disease (Liew et al., J Lab Clin Med 2006, 147:126-132). In fact, a recent study reported that PBMCs share more than 80% of the gene expression profile, or transcriptome, with many target tissues, including lung (Hansel et al., J Lab Clin Med 2005, 145:263-274). Furthermore, PBMCs have been successfully used to identify gene expression differences associated with several inflammatory or autoimmune diseases, including asthma (Bull et al., Am J Respir Crit Care Med 2004, 170:911553 919), pulmonary arterial hypertension (Bovin et al., Immunol Lett 2004, 93:217-226), and rheumatoid arthritis (Cui et al., Cancer Res 2001, 61:4947-4950). Based upon the foregoing, and the fundamental link between DNA methylation and gene transcription, PBMCs are employed to identify methylation changes potentially underlying the pathophysiological or mechanistic basis of COPD.

### Association Analysis

Association analysis by OLS regression of each of the four lung function or decline measures with the selected CpG sites yields minimum p-values of 0.00135, 0.00094, 0.00009 and 0.00343, with minimum corresponding q-values of 0.250, 0.215, 0.053 and 0.335, for age-decline, pack-years decline, CPD x age-decline, and baseline lung function, respectively. Choosing a q-value cutoff of 0.3 to isolate significant associations, 31 CpG sites associating with age-decline (p-values ranged from 1.34×10⁻³ to 0.015), 45 CpG sites associating with pack-years decline (p-values ranged from 9.42×10⁻⁴ to 0.022), 1 CpG site associating with CPD x age-decline (p=8.63×10−5), and 0 CpG sites associating with baseline lung function are identified.

CPD x Age-Decline Association. Although only one CpG site, CCR5_P630_R, (SEQ ID NO: 9) which is found in the Homo sapiens chemokine (C-C motif) receptor 5 (CCR5) gene (see NCBI Reference Sequence: NM_{—}000579 (version NM_{—}000579.1) SEQ ID NO: 73) is significantly associated with the CPD x age-decline measure, it yielded the smallest p-value (p=8.63×10-5, q=297 0.053) and thus likely represents one of the most significant sites identified. CCR5_P630_R maps to the gene encoding chemokine (C-C motif) receptor 5 (CCR5) which has been primarily studied for its role as an HIV co-receptor ((Mohr et al., Trends Mol Med 2007, 13:422-432), but has also been linked in recent years to COPD. CCR5-deficient mice have reduced levels of the cigarette smoke-induced pulmonary inflammation that is characteristic of COPD (Smyth et al., Clin Exp Immunol 2008, 154:56-63). Furthermore, CCR5 expression is shown to correlate with COPD severity (Costa et al., Chest 2008, 133:26-33), and the CCR5 chemokine CCL5 is increased in sputum from COPD patients relative to non-smokers (Donnelly et al., Trends Pharmacol Sci 2006, 27:546-553), as well as in lung explants of COPD patients compared with non-COPD smokers (Costa et al., Chest 2008, 133:26-33). The results provide mechanistic insights as methylation changes at the CCR5 gene likely influence expression levels and may be at least partially responsible for the abnormal inflammatory response observed in COPD. Furthermore, this knowledge indicates additional novel therapeutic anti-inflammatory interventions to those already under investigation for COPD (Jin et al., Cytokine 2008, 44:1-8; Vogel et al., Cell Signal 2006, 18:1108-1116).

Pack-Years Decline Associations.

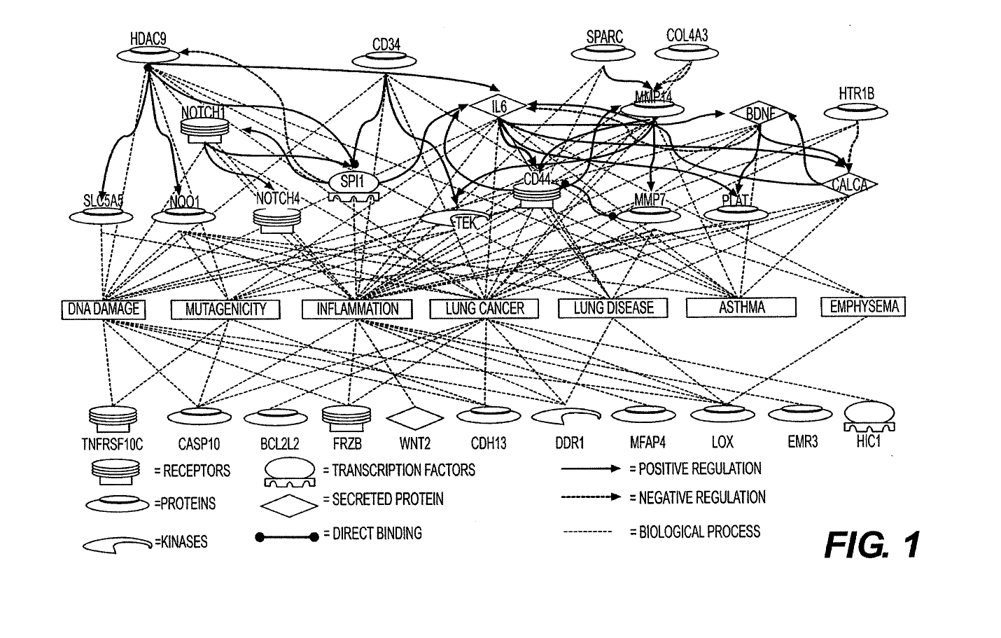

Forty-five methylation sites are significantly associated with the pack-years decline lung function measure (Table 2). Seven of these methylation sites (in bold, Table 2) are also significantly linked with the age-decline lung function measure (discussed in more detail below). Three genes (HTR1B, MFAP4, and WNT2, see SEQ ID NOs 74, 76, and 81) are each represented by two independent methylation sites, and two different Notch homologs (NOTCH1 and NOTCH4, see SEQ ID NOs 77 and 78) are also significantly associated with the pack-years decline lung function measure. Of the 41 unique genes represented in this list, 18 interact to form a network in which each gene is linked to one or more network genes (FIG. 1). Using Pathway Studio to identify and visualize links to the disease areas and biopathological mechanisms commonly associated with COPD revealed many links to oxidative stress-related mechanisms (DNA damage, mutagenicity), inflammation, and pulmonary disorders (lung cancer, lung disease) (FIG. 1). An additional 11 of the 41 identified genes also are linked to one or more of these same pulmonary disorders.

TABLE 2
Methylation (CpG) sites significantly associated (q < 0.3) with the Pack-
years decline lung function measure. Sites also significantly associated with
the age-decline lung function measure are in bold and marked with an “*”.

| | | NCBI Reference | | |
|---|---|---|---|---|
| | SEQ ID | Sequence ID and | | |
| CpG site | NO: | Version | Gene Name | Product |
| ACVR1C_P363_F * | 1 | NM_145259.1 | ACVR1C | activin A receptor, type IC |
| ATP10A_P147_F * | 3 | NM_024490.2 | ATP10A | ATPase, Class V, type 10A |
| BCL2L2_P280_F | 4 | NM_004050.2 | BCL2L2 | BCL2-like 2 protein |
| BDNF_P259_R | 5 | NM_170733.2 | BDNF | brain-derived neurotrophic |
| | | | | factor isoform a preproprotein |
| CALCA_E174_R | 6 | NM_001033952.1 | CALCA | calcitonin isoform CALCA |
| | | | | preproprotein |
| CASP10_E139_F | 7 | NM_001230.3 | CASP10 | caspase 10 isoform a |
| | | | | preproprotein |
| CASP10_P334_F | 8 | NM_001230.3 | CASP10 | caspase 10 isoform a |
| | | | | preproprotein |
| CD34_P780_R | 10 | NM_001025109.1 | CD34 | CD34 antigen isoform a |
| CD44_P87_F | 11 | NM_001001389.1 | CD44 | CD44 antigen isoform 2 |
| | | | | precursor |
| CDH13_E102_F | 12 | NM_001257.3 | CDH13 | cadherin 13 preproprotein |
| COL4A3_P545_F | 14 | NM_031366.1 | COL4A3 | alpha 3 type IV collagen |
| | | | | isoform 5, precursor |
| DDR1_E23_R | 15 | NM_001954.3 | DDR1 | discoidin domain receptor |
| | | | | family, member 1 isoform b |
| EMR3_E61_F | 18 | NM_152939.1 | EMR3 | egf-like module-containing |
| | | | | mucin-like receptor 3 isoform b |
| FRZB_E186_R | 20 | NM_001463.2 | FRZB | frizzled-related protein |
| GABRB3_P92_F | 21 | NM_021912.2 | GABRB3 | gamma-aminobutyric acid |
| | | | | (GABA) A receptor, beta 3 |
| | | | | isoform 2 precursor |
| GRB10_P496_R | 22 | NM_001001555.1 | GRB10 | growth factor receptor-bound |
| | | | | protein 10 isoform c |
| HDAC9_P137_R | 23 | NM_014707.1 | HDAC9 | histone deacetylase 9 isoform 3 |
| HIC-1_seq_48_S103_R | 24 | NM_006497.2 | HIC1 | hypermethylated in cancer 1 |
| HS3ST2_E145_R | 26 | NM_006043.1 | HS3ST2 | heparan sulfate D- |
| | | | | glucosaminyl 3-O |
| | | | | sulfotransferase 2 |
| HTR1B_E323_R | 27 | NM_000863.1 | HTR1B | 5-hydroxytryptamine |
| | | | | (serotonin) receptor 1B |
| | | | | (HTR1B_E232_R methylation |
| | | | | site for Homo sapiens 5- |
| | | | | hydroxytryptamine (serotonin) |
| | | | | receptor 1B (HTR1B)) |
| HTR1B_P222_F * | 28 | NM_000863.1 | HTR1B | 5-hydroxytryptamine |
| | | | | (serotonin) receptor 1B |
| IL6_E168_F | 30 | NM_000600.1 | IL6 | interleukin 6 (interferon, beta 2) |
| KIAA1804_P689_R * | 31 | NM_032435.1 | KIAA1804 | mixed lineage kinase 4 |
| LMO2_P794_R | 32 | NM_005574.2 | LMO2 | LIM domain only 2 |
| LOX_P313_R | 33 | NM_002317.3 | LOX | lysyl oxidase preproprotein |
| MATK_P190_R | 34 | NM_139355.1 | MATK | megakaryocyte-associated |
| | | | | tyrosine kinase isoform a |
| MFAP4_P10_R | 36 | NM_002404.1 | MFAP4 | microfibrillar-associated |
| | | | | protein 4 |
| MFAP4_P197_F | 37 | NM_002404.1 | MFAP4 | microfibrillar-associated |
| | | | | protein 4 |
| MMP14_P13_F | 38 | NM_004995.2 | MMP14 | matrix metalloproteinase 14 |
| | | | | preproprotein |
| MMP7_E59_F | 39 | NM_002423.3 | MMP7 | matrix metalloproteinase 7 |
| | | | | preproprotein |
| NOTCH1_P1198_F | 42 | NM_017617.2 | NOTCH1 | notch1 preproprotein |
| NOTCH4_E4_F | 43 | NM_004557.3 | NOTCH4 | notch4 preproprotein |
| NQO1_P345_R | 45 | NM_001025434.1 | NQO1 | NAD(P)H menadione |
| | | | | oxidoreductase 1, dioxin- |
| | | | | inducible isoform c |
| PALM2- | 47 | NM_147150.1 | PALM2- | PALM2-AKAP2 protein |
| AKAP2_P183_R | | | AKAP2 | isoform 2 |
| PLAT_E158_F | 49 | NM_000931.2 | PLAT | plasminogen activator, tissue |
| | | | | type isoform 2 precursor |
| SLC5A5_E60_F | 57 | NM_000453.1 | SLC5A5 | solute carrier family 5 (sodium |
| | | | | iodide symporter), member 5 |
| SOX1_P294_F * | 59 | NM_005986.2 | SOX1 | SRY (sex determining region |
| | | | | Y)-box 1 |
| SPARC_P195_F | 60 | NM_003118.2 | SPARC | secreted protein, acidic, |
| | | | | cysteine-rich (osteonectin) |
| SPI1_P48_F | 61 | NM_003120.1 | SPI1 | spleen focus forming virus |
| | | | | (SFFV) proviral integration |
| | | | | oncogene spi1 |
| TEK_P479_R | 63 | NM_000459.1 | TEK | TEK tyrosine kinase, |
| | | | | endothelial |
| TNFRSF10C_P612_R | 64 | NM_003841.2 | TNFRSF10C | tumor necrosis factor receptor |
| | | | | superfamily, member 10c |
| | | | | precursor |
| TRIP6_P1274_R * | 66 | NM_003302.1 | TRIP6 | thyroid hormone receptor |
| | | | | interactor 6 |
| WNT2_E109_R | 68 | NM_003391.1 | WNT2 | wingless-type MMTV |
| | | | | integration site family member |
| | | | | 2 precursor |
| WNT2_P217_F | 69 | NM_003391.1 | WNT2 | wingless-type MMTV |
| | | | | integration site family member |
| | | | | 2 precursor |
| ZMYND10_P329_F | 70 | NM_015896.2 | ZMYND10 | zinc finger, MYND domain- |
| | | | | containing 10 |

A more detailed analysis of the 41 genes and their functional roles revealed three additional common themes. Several genes encode, interact with, or remodel components of the extracellular matrix. These include the collagen subunit COL4a3, the secreted structural protein SPARC, which is considered a potential component of collagen, and the collagen binding proteins CD44 and MFAP4. Additionally, lysyl oxidase (LOX), an enzyme involved in Extra Cellular Matrix (ECM) assembly is included in this gene set, as are several genes associated with ECM breakdown, including two matrix metalloproteinases (MMP7 and MMP14), tissue plasminogen activator PLAT, and DDR1, which is a collagen-activated receptor tyrosine kinase that is thought to modulate ECM breakdown by way of MMP activation (Terstappen et al., Blood 1991, 77:1218-1227).

The second common theme to emerge relates to an additional subset of these 41 genes which is involved in haematopoesis. Included in this set are CD34, a cell surface antigen found on haematopoetic stem cells (Jönsson et al., Eur J Immunol 2001, 31:3240-3247), two Notch homolog cell surface receptors (NOTCH1(Ye et al., Leukemia 2004, 18:777-787) and NOTCH4 (Takakura et al., Immunity 1998, 9:677-686)) that are expressed at different stages of haematopoesis, and the receptor tyrosine kinase TEK (Nam et al., Mol Ther 2006, 13:15-25). Additionally, important transcriptional regulators LMO2, a key regulator of early haematopoetic development (Ivascu et al., Int J Biochem Cell Biol 2007, 39:1523-1538), and SPI1, which has recently been shown to be differentially methylated in different cell lineages and stages of the haematopoetic cascade (Petrie et al., J Biol Chem 2003, 278:16059-16072), are also included in this gene subset, as are haematopoesis-linked histone deacetylase HDAC9 (Avraham et al., J Biol Chem 1995, 270:1833-1842) and signaling protein MATK (Nemeth et al., Cell Res 2007, 17:746-758).

The third subset of genes associated with the pack-years decline lung function measure shares common links to the Wnt-signalling pathway and include the secreted glycoprotein WNT2 (Bovolenta et al., J Cell Sci 2008, 121:737-746), as well as Wnt-antagonists ERZB (Tezuka et al., Biochem Biophys Res Commun 2007, 356:648-654) and GRB10 (Zhai et al., Am J Pathol 2002, 160:1229-1238). In addition, two Wnt-regulated targets, the matrix metalloproteinase MMP7 (Ayyanan et al., Proc Natl Acad Sci USA 2006, 103:3799-3804) and NOTCH4 homolog (Marciniak et al., Thorax 2009, 64:359-364) receptor, are also found in this subset. Finally, the receptor tyrosine kinase DDR1 is thought to receive lateral signaling input from Wnt355 ligand/receptor complexes (Terstappen et al., Blood 1991, 77:1218-1227).

The observation linking ECM-associated genes with the pack-years decline lung function measure is significant given that ECM integrity in alveolar tissue is increasingly recognized as a key player in COPD pathogenesis (Pavlisa et al., Clin Sci (Lond) 2004, 106:43-51). Accordingly, 6 of these 8 genes have been previously linked to COPD. The haematopoetic link could reflect an impaired response to hypoxia in COPD. Clinical work has shown that patients suffering from severe lung disease, including COPD, exhibit impaired hematological response to hypoxia (Fadini et al., Stem Cells 2006, 24:1806-1813) with reduced levels of all circulating blood progenitor cells (Karrasch et al., Respir Med 2008, 102:1215-1230). The Wnt signaling pathway has not previously been linked to COPD, but it has been linked to inflammation and oxidative stress.

Age-Decline Associations.

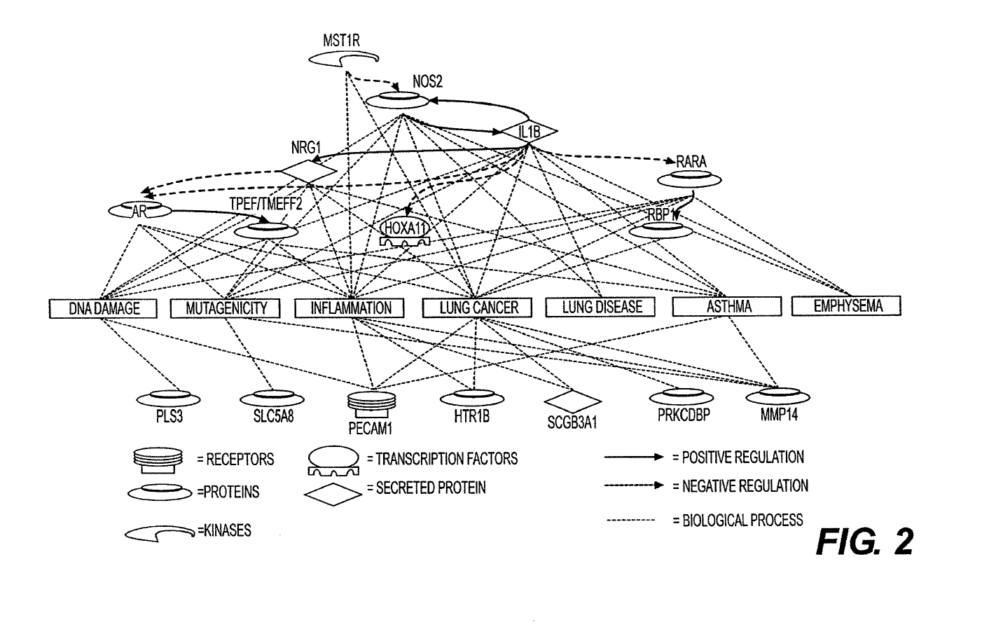

The aging process is recognized as an important contributor to the development and progression of COPD (Ito et al., Chest 2009, 135:173-180; Uchida et al., Biochem Biophys Res Commun 1999, 266:593-602). Although epigenetic mechanisms are thought to be at least partially responsible for this link, little is known regarding the underlying specific molecular processes at work. In the analysis, 31 CpG sites are significantly associated with the age-decline lung function measure (Table 3). Nine of the 31 genes mapping to these methylation sites form an interaction network and are linked to at least one of the same COPD-associated disease areas or biopathological mechanisms described for significant pack-years decline associations (FIG. 2). An additional 7 genes are linked to at least one of these same disease areas.

TABLE 3
Methylation (CpG) sites significantly associated (q < 0.3) with the age-decline lung function measure.

| | | NCBI Referenc | | |
|---|---|---|---|---|
| | SEQ ID | Sequence ID and | | |
| CpG site | NO: | Version | Gene Name | Product |
| ACVR1C_P363_F | 1 | NM_145259.1 | ACVR1C | activin A receptor, type IC |
| AR_P54_R | 2 | NM_001011645.1 | AR | androgen receptor isoform 2 |
| ATP10A_P147_F | 3 | NM_024490.2 | ATP10A | ATPase, Class V, type 10A |
| CDK10_P199_R | 13 | NM_052987.2 | CDK10 | cyclin-dependent kinase |
| | | | | 10 isoform 2 |
| DKFZP564O0823_P386_F | 16 | NM_015393.2 | DKFZP564O0823 | DKFZP564O0823 protein |
| DLC1_E276_F | 17 | NM_182643.1 | DLC1 | deleted in liver cancer 1 |
| | | | | isoform 1 |
| ERG_E28_F | 19 | NM_004449.3 | ERG | v-ets erythroblastosis |
| | | | | virus E26 oncogene like |
| | | | | isoform 2 |
| HOXA11_P698_F | 25 | NM_005523.4 | HOXA11 | homeobox protein A11 |
| HTR1B_P222_F | 27 | NM_000863.1 | HTR1B | 5-hydroxytryptamine |
| | | | | (serotonin) receptor 1B |
| IL1B_P582_R | 29 | NM_000576.2 | IL1B | interleukin 1, beta |
| | | | | proprotein |
| KIAA1804_P689_R | 31 | NM_032435.1 | KIAA1804 | mixed lineage kinase 4 |
| MEST_E150_F | 35 | NM_002402.2 | MEST | mesoderm specific |
| | | | | transcript isoform a |
| MMP14_P13_F | 36 | NM_004995.2 | MMP14 | matrix metalloproteinase |
| | | | | 14 preproprotein |
| MST1R_E42_R | 40 | NM_002447.1 | MST1R | macrophage stimulating 1 |
| | | | | receptor |
| NOS2A_E117_R | 41 | NM_000625.3 | NOS2A | nitric oxide synthase 2A |
| | | | | isoform 1 |
| NPR2_P1093_F | 44 | NM_003995.3 | NPR2 | natriuretic peptide |
| | | | | receptor B precursor |
| NRG1_P558_R | 46 | NM_013958.1 | NRG1 | neuregulin 1 isoform |
| | | | | HRG-beta3 |
| PECAM1_P135_F | 48 | NM_000442.2 | PECAM1 | platelet/endothelial cell |
| | | | | adhesion molecule (CD31 |
| | | | | antigen) |
| PLS3_E70_F | 50 | NM_005032.3 | PLS3 | plastin 3 |
| PRKCDBP_E206_F | 51 | NM_145040.2 | PRKCDBP | protein kinase C, delta |
| | | | | binding protein |
| RAB32_P493_R | 52 | NM_006834.2 | RAB32 | RAB32, member RAS |
| | | | | oncogene family |
| RARA_P1076_R | 53 | NM_000964.2 | RARA | retinoic acid receptor, |
| | | | | alpha isoform a |
| RBP1_E158_F | 54 | NM_002899.2 | RBP1 | retinol binding protein 1, |
| | | | | cellular |
| SCGB3A1_E55_R | 55 | NM_052863.2 | SCGB3A1 | secretoglobin, family 3A, |
| | | | | member 1 |
| SEPT5_P464_R | 56 | NM_001009939.1 | SEPT5 | septin 5 isoform 2 |
| SLC5A8_E60_R | 57 | NM_145913.2 | SLC5A8 | solute carrier family 5 |
| | | | | (iodide transporter), |
| | | | | member 8 |
| SOX1_P294_F | 59 | NM_005986.2 | SOX1 | SRY (sex determining |
| | | | | region Y)-box 1 |
| TDGF1_P428_R | 62 | NM_003212.1 | TDGF1 | teratocarcinoma-derived |
| | | | | growth factor 1 |
| TPEF_seq_44_S36_F | 65 | NM_016192.2 | TMEFF2 | transmembrane protein |
| | | | | with EGF-like and two |
| | | | | follistatin-like domains 2 |
| TRIP6_P1274_R | 66 | NM_003302.1 | TRIP6 | thyroid hormone receptor |
| | | | | interactor 6 |
| TUSC3_E29_R | 67 | NM_178234.1 | TUSC3 | tumor suppressor |
| | | | | candidate 3 isoform b |

A detailed analysis of these genes and the function of their associated protein products revealed additional common mechanisms. In particular, inflammatory, endocrine related, and retinol signaling genes stand out. The inflammation-associated genes included the macrophage stimulating factor (MST1R), the cytokine-induced nitric oxide synthase 2 (NOS2), and the cytokine interleukin 1β (IL1β). In addition, several genes are linked to the growth factor cytokine TGFβ, including TPEF/TMEFF2 which is thought to bind and inactivate TGFβ (Gendron et al., Biol Reprod 1997, 56:1097-1105), the ALK7/ACVR1C receptor that binds the TGFβ-family of ligands, and TDGF1 that is regulated by TGFβ.

Among endocrine-related genes in this subset, AR, TRIP6 and NPR2 are all hormone receptors, binding androgen hormone, thyroid hormone, and natriuretic peptide, respectively. Additionally, HOXA11 is a transcription factor involved in reproductive development (Eun Kwon et al., Ann N Y Acad Sci 2004, 1034:1-18) and its expression increases during implantation due to sex steroid hormones (Lacroix-Fralish et al., Neuron Glia Biol 2006, 2:227-234). Similarly, the signaling molecule neuregulin 1 (NRG1) has also been shown to be regulated by sex steroid hormones (Gery et al., Oncogene 2002, 21:4739-4746), as has TPEF/TMEFF2 whose expression is androgen-induced (Nilsson et al., Crit Rev Toxicol 2002, 32:211-232).

Two genes, RBP1 and RARA, significantly associated with the age-decline lung function measure, are responsible for retinol signaling. The retinol binding protein RBP1 is the carrier protein responsible for the transport of retinol from the liver to peripheral tissue. After retinol binding protein-mediated transport of retinol and cellular uptake, retinol can be converted intracellularly to retinoic acid, which can translocate to the nucleus. Retinoic acid then binds the nuclear retinoic acid receptor RARA, triggering a cascade of transcriptional events leading to the regulation of specific target genes (Barnes P J. J Clin Invest 2008, 118:3546-3556).

Inflammation is recognized as being of importance in COPD (Van Vliet et al., Am J Respir Crit Care Med 2005, 172:1105-1111) and a number of inflammatory genes are shown herein to be associated with the age-decline lung function measure. As shown in FIG. 2, inflammation may be influencing the other identified processes in this network through IL1β links to endocrine-related and retinol signaling genes. Furthermore, the TPEF/TMEFF2 gene represents another common link as it appears to factor into inflammatory processes through its likely interaction with TGFβ while also being regulated by androgen. Endocrine system dysfunction has been linked to COPD (Andreassen et al., Eur Respir J Suppl 2003, 46:2s-4s) yet the underlying mechanisms remain poorly understood (Hind et al., Thorax 2009, 64:451-457). The results presented herein provide novel insights into the specific pathways and molecular mechanisms underlying this aspect of COPD pathophysiology. Retinol signaling has been previously implicated in COPD; investigations of the therapeutic value of retinoic acid treatment show mixed results in animal and human studies.

Associations Summary.

Using the high-throughput GoldenGate® DNA methylation assay on DNA samples extracted from PBMCs of 311 cigarette smokers with or without COPD, it is observed that 71 CpG sites, corresponding to 67 unique genes, is significantly associated with one or more lung function decline measures. These CpG sites represent novel DNA methylation biomarkers for risk or progression of smoking-associated COPD that can be readily detected in the blood and which may facilitate early diagnosis and prognostic ability in COPD.

### Example 2

### Statistical Method for Excluding Non-Variable CpG Sites in High-Throughput DNA Methylation Profiling

A method to estimate the proportion of non-variable CpG sites and exclude those sites from further analysis is disclosed. The method employs correlations between technical replicates obtained by assaying the same samples twice. This is illustrated by analyzing methylation profiles generated using DNA extracted from the PBMCs of 311 human subjects.

Although excluding non-variable CpG sites is relevant in all instances, it may be particularly important for peripheral biofluids, such as blood. Peripheral biofluids are often analyzed when it is not feasible to obtain diseased target tissue. Furthermore, methylation markers that can be measured in peripheral biofluids are potentially much better for diagnostic and prognostic purposes because of the relatively simple, non-invasive manner in which the biosamples can be collected. There is a considerable amount of evidence showing that methylation markers are not limited to the affected tissue or cell type, but can be detected in peripheral biofluids. A clear example involves loss of imprinting of IGF2, which is found in the colon as well as lymphocytes and where either methylation marker is associated with increased colorectal cancer risk.

Two factors may explain why methylation markers can be detected in peripheral biofluids. First, peripheral blood-based studies may be useful in revealing methylation changes predating or resulting from the epigenetic reprogramming events affecting the germ line and early embryogenesis (Rakyan et al., Biochem. J. 2001, 356:1-10; Yeivin et al., (2008) Gene methylation patterns and expression. In Jost, J. and Saluz, H. (eds), DNA methylation: molecular biology and biological significance. Birkhauser-Verlag, Basel, pp. 523-568; Efstratiadis, A. (1994) Curr. Opin. Genet. Dev., 4, 265-280; Monk, et al., (1987) Development, 99, 371-382). As the epigenetic profile of somatic cells is mitotically inherited, these epigenetic mutations can be found in cells from peripheral blood. Second, blood contains proteins, metabolites, cells that have been modified as they circulate through diseased tissues and cell-free DNA from diseased tissues and cells. As such, traces of the aberrant methylation in diseased target tissue may be present in peripheral biofluids. The problem here, however, is that methylation markers in peripheral biofluids will not uniquely reflect the physiological and pathophysiological state of the relevant disease tissues. This fact can potentially reduce the ability to detect biological variation in methylation status, and further highlights the need to filter non-variable probes prior to conducting disease or phenotype association tests. Employing suitable filters improves the statistical power to detect biologically meaningful results.

### Probe Correlations.

To evaluate the magnitude of the methylation signal versus the measurement error, methylation status on each biosample was measured twice. Assume that the methylation measurement y for biosample i, i=1 . . . N, on probe j, j=1 . . . K, is a function of the true methylation status plus a measurement error that may be caused by factors related to sample preparation, image processing, or similar technical issues:

yᵢⱼ₍₁₎=mᵢⱼ+eᵢⱼ₍₁₎

yᵢⱼ₍₂₎=mᵢⱼ+eᵢⱼ₍₂₎

where mᵢⱼ is the true methylation status of a sample of biological material containing DNA (aka a “biosample”) for i on probe j, and eᵢⱼ is the measurement error for biosample i on probe j. Subscripts 1 and 2 are used to distinguish the two measurement occasions. Note that mᵢⱼ is not subscripted as it is expected the methylation status will remain unchanged on the two occasions.

If it is assumed that the measurement errors are uncorrelated, COV(eᵢⱼ₍₁₎, eᵢⱼ₍₂₎)=0, the covariance between the measured methylation signals across the two occasions equals the variance of the true methylation signals for probe j: COV(yᵢⱼ₍₁₎, yᵢⱼ₍₂₎)=VAR(Mⱼ). Mⱼ includes true methylation status of all biosamples for probe j and equals {m₁ⱼ, m₂ⱼ, . . . , m_{Nj}}. Furthermore, if it is assumed that the precision of the measurements is similar across the two occasions, VAR(eᵢⱼ₍₁₎)=VAR(eᵢⱼ₍₂₎)=VAR(Eⱼ), then the variance of the measured methylation signals equals VAR(yᵢⱼ₍₁₎)=VAR(yᵢⱼ₍₂₎)=VAR(Yⱼ)=VAR(Mⱼ)+VAR(Eⱼ). Consequently, the correlation for probe j across the two occasions becomes:

$$COR ( {y}_{ij ( 1 )} , {y}_{ij ( 2 )} ) = \frac{VAR ( {M}_{j} )}{VAR ( {M}_{j} ) + VAR ( {E}_{j} )} \qquad ( 1 )$$

This probe correlation is an index of the signal-to-error ratio, as it equals the true methylation variance divided by the total variance that includes the error variance as well.

Equation (1) implies that probe correlations can be low for two reasons. First, the measurement error may overwhelm the true methylation signal so that the probe mainly measures error (i.e. VAR(Eⱼ)>>VAR(Mⱼ). Second, the probe correlation may be low because there is little biological variation in methylation status among biosamples (i.e. VAR(Mⱼ)≈0). To explore the two possibilities, the sample correlations as well as the correlation between all probe correlations and the corresponding probe variances can be examined.

The sample correlations are calculated after first transposing the data matrix so that the K probes are now in the rows and biosamples in the columns. In this transposed data matrix, yᵢⱼ is the methylation measurement for probe j on biosample i. Using assumptions similar to those upon which Equation (1) is based, the sample correlation for biosample i measured on two occasions equals:

$$COR ( {Y}_{i ( 1 )} , {Y}_{i ( 2 )} ) = \frac{VAR ( {M}_{i} )}{VAR ( {M}_{i} ) + VAR ( {E}_{i} )} \qquad ( 2 )$$

where VAR(Mᵢ) is the variance in true methylation status across all probes and VAR(Eᵢ) is the variance in the measurement error across all probes for biosample i. If measurement error is large relative to differences among probes in their methylation status, in addition to observing low probe correlations, a low sample correlation would be expected. In contrast, the combination of low probe correlations and high sample correlations suggests little variation in true methylation across biosamples.

A second way to examine whether low probe correlations are caused by large error variances as opposed to low variances in true methylation status uses all probes to calculate the correlations between technical replicate probe correlations and the total probe variances. If the probe correlation is low primarily due to large measurement errors, a negative correlation between the probe correlations and the total probe variances is expected. This stems from the observation that probes with large error variance, VAR(Eⱼ), will on average have large total variance because VAR(Yⱼ)=VAR(Mⱼ)+VAR(Eⱼ), but lower probe correlations, as follows from Equation (1). On the other hand, if probe correlations are low because of low variances in true methylation status a positive correlation would be expected. This is because probes with larger variation in true methylation signal, VAR(Mⱼ), will on average have larger total variance, VAR(Yⱼ), in addition to larger probe correlations according to Equation (1).

### Mixture Modeling.

Although the above analyses enable researchers to get a general sense of the magnitude of the true methylation status versus the measurement error, it does not provide specific guidelines about which individual probes to include in further analysis. For that purpose an analysis of all the probe correlations using a mixture model would be more accurate. In the mixture model, the distribution of the probe correlations is assumed to be a function of discrete underlying distributions. The number of underlying distributions can be determined empirically. In the simplest case of two distributions, one of the underlying distributions may represent probes showing little variation in true methylation status across biosamples whereas the other may represent probes showing substantial variation in true methylation status across biosamples. Based on the estimated mixture model an estimate of the (posterior) probability of each probe belonging to each class can be obtained. These posterior probabilities can subsequently be used for probe selection.

MATLAB® (The MathWorks, Inc., Natick, Mass.) was used to estimate mixture models. MATLAB® uses the Expectation-Maximization algorithm (EM) to estimate the parameters of the mixture model. In the Expectation step, the posterior probability of each probe is calculated using the current model parameters (i.e. the mixing proportions, means, and variances). In the Maximization step, the model parameters are estimated using the current posterior probabilities. The cycle of Expectation and Maximization steps is repeated until convergence is achieved. Technical details of the model can be found in the material below, particularly in EXAMPLE 3 “Fitting of a Two-Class Mixture Model to the Probe Correlations”.

### Application to Illumina Goldengate Methylation Array Subjects, Biosamples and Methylation Data Regeneration

DNA is extracted from whole blood samples from 311 middle-aged and older males and females who had participated in the LHS (Anthonisen et al. (1994) JAMA, 272, 1497-1505; Connett et al. (1993) Control. Clin. Trials, 14, 3S-19S) and GAP at the University of Utah. Of the 311 subjects, 145 are cigarette smokers with spirometrically defined COPD (Rabe et al., 2007), and 166 did not have COPD (91 never smokers and 75 smokers).

The GoldenGate® Assay for Methylation (Illumina Inc., San Diego, Calif.) is used to assess the DNA methylation status of 1,505 CpG sites from 807 genes, simultaneously. Prior to methylation profiling, bisulfite conversion of the DNA biosamples is conducted using the EZ DNA Methylation Kit™ (Zymo Research Corp., Orange, Calif.) in a 96-well format; as per the manufacturer's protocol; 2 μg of genomic DNA is used for bisulfite conversion. Following conversion, 250 ng of DNA is used for the methylation assay. The BeadStudio® Methylation Module (Illumina Inc., San Diego, Calif.) is used to read fluorescent signals from scanned images collected from the Illumina Beadarray® Reader.

The 311 DNA biosamples are analyzed using five Illumina GoldenGate® matrices. Technical replicates are obtained for 126 biosamples by analyzing each on two separate matrices. The methylation status of each CpG site is calculated based on fluorescent intensities corresponding to the methylated allele (Cy5) and the unmethylated allele (Cy3). In order to remove measurement artifacts prior to calculating the methylation status, Cy3 and Cy5 fluorescent intensities are independently corrected for background signal, as well as for differential bisulfite conversion levels between biosamples using an OLS regression model. Following signal correction, the methylation measurement y for biosample i on probe j is calculated as the ratio of fluorescent intensities from the methylated allele (Cy5) to the total fluorescent signal from both the methylated allele (Cy5) and the unmethylated allele (Cy3) such that:

$${y}_{ij} = \frac{Cy {5}_{ij}}{Cy {5}_{ij} + Cy {3}_{ij}} \qquad ( 3 )$$

Because this quantity is a ratio, yᵢⱼ is a continuous number between 0 and 1. Complete technical details for Cy3 and Cy5 corrections and yᵢⱼ calculations are provided below, particularly in the section titled “Methylation Status”.

### Association Analyses.

The outcomes in this analysis are four measures of lung function or decline in lung function measured spirometrically as FEV₁ (Knudson et al. (1983) Am. Rev. Respir. Dis., 127, 725-734). These four measures are derived by fitting mixed models to longitudinal spirometric, smoking history, and demographic data obtained over the subjects' 17-year average participation period in the LHS and GAP. Conceptually, these measures represent different underlying biological processes driving lung function decline. This embodiment focused on age-related decline (age-decline), pack-years-related decline (pack-years decline), the intensifying effects of smoking, in terms of number of cigarettes per day (CPD) and decline with age (CPD x age-decline) that together accounted for the vast majority of individual differences in lung function decline in these subjects. In addition, this embodiment included baseline lung function measured at subjects' entry into the study as an outcome measure as it has also been shown to vary in magnitude across individuals (Griffith, K. A. et al. (2001) Am. J. Respir. Crit. Care Med., 163, 61-68). Technical details for the outcome variables are provided in the materials below, especially the section “Measures of Lung Function and Decline.

To test for association between DNA methylation variables and lung function decline outcome variables, regression analyses is performed with the probes as predictor variables. The F-test statistic is used to perform significance tests. Separate analyses are conducted on all probes as well as on only the subset of probes that remained after selection. Two criteria are used to evaluate the performance of the probe selection method. First, the proportion of markers without effect (p₀) is estimated using the estimator proposed by Meinshausen and Rice (Meinshausen et al., (2006) Ann. Stat., 34, 373-393), which performs well in scenarios where p₀ is close to one. Thus, after successful probe selection, this embodiment would expect a smaller proportion of markers without effects. Second, the distribution of q-values (Storey J: The Annals of Statistics 2003, 31:2013-2035; Storey et al., Proc Natl Acad Sci USA 2003, 100:9440-9445) is examined. These q-values are positive false discovery rates (pFDRs) calculated by using the p-value of the markers as the threshold for declaring significance. Successful probe selection results in more significant results across a range of previously specified q-value thresholds used to declare significance.

### Probe Selection.

Probe correlations are calculated using the 126 replicate biosamples. The mean of probe correlations across the 1,505 probes is 0.268 (SD=0.246). This suggested that, on average, sample differences in methylation status accounted for only 26.8% of the total variation. Equation (1) indicates two possible reasons for the low probe correlations. First, VAR(Eⱼ) may be much larger than VAR(Mⱼ) so that the true methylation signals are overwhelmed by the measurement error. Alternatively, VAR(Mⱼ), the methylation difference among biosamples, may be close to zero.

To explore whether large error variance versus limited variation in methylation signal caused the small probe correlations, this embodiment first calculated the sample correlation defined in Equation (2). In sharp contrast to the probe correlations, the sample correlations calculated using the 126 replicate biosamples are high, with a mean of 0.995 (SD=0.0037). The high sample correlations indicate that the measurement errors are relatively small compared with the methylation variations among probes, because large measurement errors would yield large denominators in Equation (2) and result in low sample correlations. Accordingly, the high sample correlations observed suggest that the low probe correlations are not caused by large measurement errors but rather reflect low variation in methylation among the individuals studied.

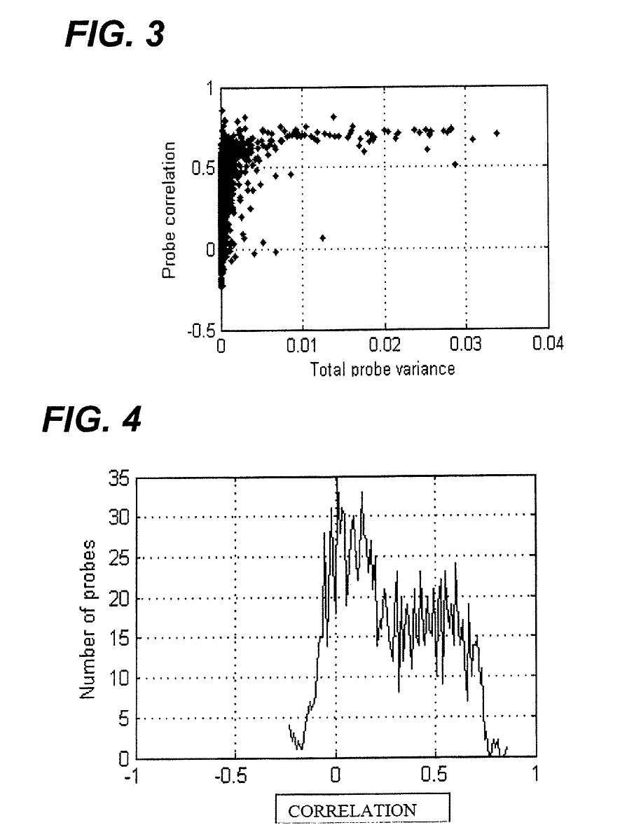

This embodiment then analyzed the correlation between the 1,505 probe correlations and the 1,505 total probe variances. As shown in FIG. 3, probes with high probe correlations also have a relatively large total variance. This observation also supports the idea that low probe correlations are primarily due to low methylation-related variation among biosamples rather than large measurement errors.

This embodiment then attempted to determine which probes should be removed prior to conducting the subsequent statistical analyses. FIG. 4 shows the distribution of 1,505 probe correlations. The bi-modality indicated in the figure suggested that probes may fall into two different classes, one with little methylation variation and low probe correlation, and the other with more methylation variation and relatively high probe correlation. Based on this plot this embodiment fitted a two-class mixture model. The first class had an estimated mean probe correlation of 0.51 (SD=0.019) with a mixing proportion of 0.42 and the second class had an estimated mean of 0.09 (SD=0.016) with a mixing proportion of 0.58. These results indicate that nearly 60% of probes had very little variation, highlighting the significance of this probe selection problem.

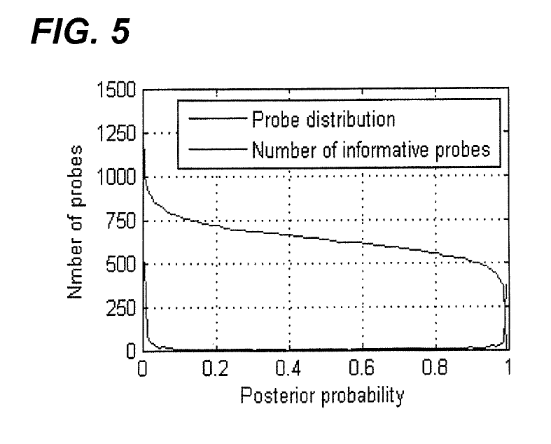

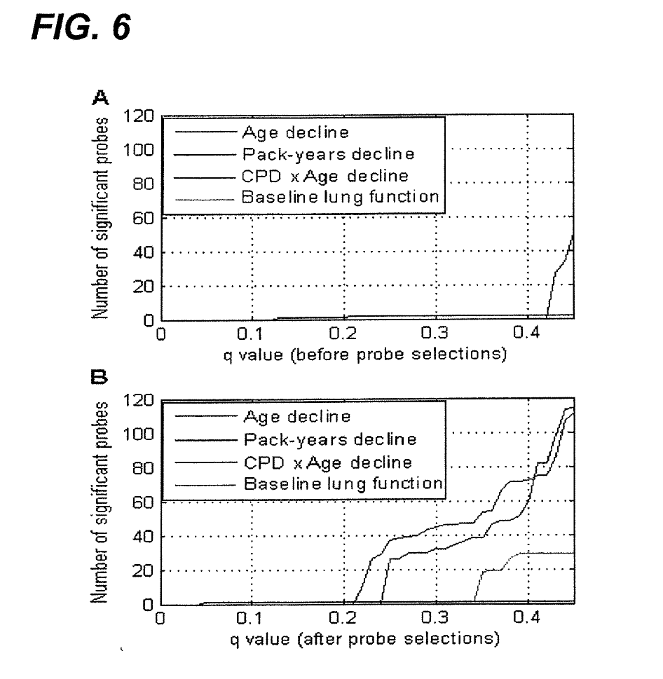

Based on the mixture model, the posterior probabilities of each probe belonging to each class are estimated. The extreme bimodal distribution of the posterior probabilities (FIG. 5) further support the validity of using a two-class mixture model in this context, and implies that most of the probes can be assigned to one or the other of the classes with reasonably high confidence. Furthermore, the observed bimodality yields the desirable property of cut-off stability where the choice of threshold does not have a major impact on the number of probes selected (FIG. 3). Accordingly, given that probes with higher correlations are more likely to reflect biologically relevant methylation variation, this embodiment selected the 634 probes with posterior probability ≧0.5 as members of the class for subsequent analyses.

TABLE 4
p₀ etimates using test results from regression analyses

| | Before probe | After probe |
|---|---|---|
| Outcome | selection | selection |
| age-decline | 0.9996 | 0.9781 |
| pack-years decline | 0.9992 | 0.9986 |
| CPD × age-decline | 0.9970 | 0.9715 |
| baseline lung function | 1.0009 | 0.9904 |

CPD, cigarettes per day.

### Example 3

### Fitting of a Two-Class Mixture Model to the Probe Correlations

A two-class mixture model was fit to probe correlations (see the data displayed in FIG. 4). For the fitting, if the symbol xⱼ is employed to represent the probe correlation COV(yⱼ₍₁₎, yⱼ₍₂₎) of probe j, j=1 . . . K, then the density function for the probe correlations is assumed to be a mixture of two classes:

f(xⱼ;a₁,μ₁,μ₂,σ₁,σ₂)=a₁g(xⱼ;μ₁,σ₁)+(1−a₁)g(xⱼ;μ₂,σ₂)

with g(μ₁, σₗ) and g(μ₂, σ₂) as two Gaussian densities with mean μ and standard deviation σ, and where a₁ is the mixing proportion subject to the constrains that 0<a₁<1.

The Expectation-Maximization (EM) algorithm was used to calculate the parameters of the mixture model. In the expectation step, the posterior probabilities for each probe xᵢ and each class were computed as:

$$prob ( class = 1 \vert {x}_{j } ) = \frac{{a}_{1} g ( {x}_{j} ; {μ}_{1} , {σ}_{1} )}{f ( {x}_{j} ; {a}_{1} , {μ}_{1} , {μ}_{2} , {σ}_{1} , {σ}_{2} )}$$

$$prob ( class = 2 \vert {x}_{j } ) = 1 - prob ( class = 1 \vert {x}_{j } )$$

In the maximization step, the mixing proportions were computed as the means of the posterior probabilities over K probes.

$${a}_{1} = \frac{1}{K} {\sum }_{j = 1}^{K} prob ( class = 1 \vert {x}_{j } )$$

$${a}_{2} = 1 - {a}_{1}$$

The means of two classes were:

$${μ}_{1} = \frac{{\sum }_{j} prob ( class = 1 \vert {x}_{j } ) {x}_{j}}{{\sum }_{j} prob ( class = 1 \vert {x}_{j } )}$$

$${μ}_{2} = \frac{{\sum }_{j} prob ( class = 2 \vert {x}_{j } ) {x}_{j}}{{\sum }_{j} prob ( class = 2 \vert {x}_{j } )}$$

The variances of two classes were:

$${σ}_{1}^{2} = \frac{{\sum }_{j} prob ( class = 1 \vert {x}_{j } ) {( {x}_{j} - {μ}_{1} )}^{2}}{{\sum }_{j} prob ( class = 1 \vert {x}_{j } )}$$

$${σ}_{2}^{2} = \frac{{\sum }_{j} prob ( class = 2 \vert {x}_{j } ) {( {x}_{j} - {μ}_{2} )}^{2}}{{\sum }_{j} prob ( class = 2 \vert {x}_{j } )}$$

The expectation and maximization steps were repeated until model parameters converged. The mixture model was estimated using the MATLAB® Statistics Toolbox 6.1 (The MathWorks, Inc., Natick, Mass.).

### Methylation Status

Prior to calculating the methylation status, fluorescent intensities (Cy3 and Cy5) were normalized to remove measurement artifacts. Illumine® provides two standard normalization methods denoted as the background normalization and average normalization method, respectively. The background normalization method subtracts a background value calculated by averaging the signals of built-in negative controls, whereas the average normalization method averages the signals across multiple arrays. However, in this study a slightly different approach was developed to capitalize on additional characteristics of the DNA methylation array. Specifically Cy3 and Cy5 fluorescent intensities are corrected independently and also corrected for differential bisulfite conversion levels across samples using an OLS regression model.

To estimate fluorescent signals in the absence of hybridization as a means to assess background signal intensity, principal components analysis (PCA) was performed on the 22 built-in negative controls. Those negative controls are probes that lack a specific target in the genome and are included on the GoldenGate® Assay for Methylation (Illumina Inc., San Diego, Calif.) for each biosample. Since the independent variables in the OLS regression model are assumed to be independent, this embodiment applied PCA to transform the 22 negative control signals into orthogonal principal components. The first 10 principal component (PC) scores (PC_{cy3} and PC_{cy5}) were selected for inclusion in the model. While each of the 10 PC scores is not likely to be required to remove the artifactual background signal, this embodiment nonetheless chose to be more inclusive given that PCs that are not predictive, will have regression model coefficients (or weights) close to zero and thus have essentially no effect on the final adjusted value. The Cy3 signals were corrected not only by Cy3 background signals but also by Cy5 background signals since the relevance was found between Cy3 signals and Cy5 background signals. In the same way, the Cy5 signals were corrected by both Cy5 and Cy3 background signals. The Cy5/Cy3 ratios of two built-in bisulfite conversion (BC) control probes also were included in the model to correct for any bisulfite conversion differences among biosamples. The resulting regression model was constructed for each methylation probe and each GoldenGate assay matrix to normalize Cy3 and Cy5 signals separately as follows:

Cy3=β₀+Σ_{i=1:10}(βᵢ×PC_{cy3i})+Σ_{j=1:10}(βⱼ×PC_{cy5j})+Σ_{k=1:2}(βₖ×BCₖ)+ε

Cy5=β₀+Σ_{i=1:10}(βᵢ×PC_{cy3i})+Σ_{j=1:10}(βⱼ×PC_{cy5j})+Σ_{k=1:2}(βₖ×BCₖ)+ε

where Cy is the fluorescent signal (either Cy3 or Cy5), β₀ is the intercept term, βᵢ are the coefficients associated with PC_{cy3i}, βⱼ are the coefficients associated PC_{cy5j}, βₖ are the coefficients associated with BCₖ, and ε is the residual.

Normalized Cy3 and Cy5 signals were calculated as the sum of the global mean of Cy3 and Cy5 for the CpG site across matrices and their residual in the above regression analysis. Cy3 signals of some probes targeting fully methylated sequences are expected to have negative signals when signals of negative controls were regressed out during the normalization. The same is true for Cy5 signals for some probes targeting fully unmethylated sequences. To avoid potential problems introduced by including negative values, Cy3 and Cy5 were adjusted such that all signals are positive and the smallest value is 0.01.

The methylation level y of each CpG site was calculated as the ratio of adjusted intensities between methylated and unmethylated alleles as follows:

$$y = \frac{Cy 5}{Cy 5 + Cy 3}$$

This quantity was then used in the subsequent probe selection and association testing procedures.

### Measures of Lung Function and Decline.

The outcome variables used in these analyses were derived from random effects in linear mixed models analyzing longitudinal spirometric, smoking history, and demographic data (Goldstein, H. (1995) Multilevel statistical models. Wiley, New York). Specifically, data was modeled for 624 cigarette smokers with COPD and aged 35-60 at baseline, followed up 7 times over approximately 17 years (1986-2004) in the LHS (Anthonisen et al., (1994) JAMA, 272, 1497-1505; Connett et al., (1993) Control. Clin. Trials, 14, 3S-19S) and its follow-on GAP; 204 GAP subjects without COPD were also examined (see Table 5 for descriptive statistics). The Optimal model of the data was selected based on likelihood ratio tests, which were used to determine the significance of each fixed and random effect parameter as it was added to the model (Willet et al., 1998. Dev. Psychopathol., 10, 395-426). After the optimal model was identified, the outcome variables were calculated as best linear unbiased predictors (BLUPs) of the random effects. Missing data were handled by multiple imputation using chained equations, with 5 datasets imputed and analyzed (Royston, P. (2005) Multiple imputation of missing values: update. S. J., 5, 527-536; Van Buuren, S. et al. (2006) J. Stat. Comput. Sim., 76, 1049-1064).

TABLE 5
Descriptive statistics of subject characteristics at study initiation*

| | Female (N = 303) | | Male (N = 525) | |
|---|---|---|---|---|
| Variables | Mean ± SD | Range | Mean ± SD | Range |
| Age (y) | 44.82 ± 8.08 | 26-60 | 46.59 ± 7.47 | 28-68 |
| FEV₁ (L) | 2.44 ± 0.52 | 1.18-3.93 | 3.16 ± 0.63 | 1.02-6.09 |
| Height (cm) | 164.01 ± 5.88 | 150-180 | 176.89 ± 6.37 | 151-197 |
| Pack-years | 28.41 ± 20.44 | 0-87.5 | 38.14 ± 23.29 | 0-153 |
| CPD | 0.58 ± 0.60 | 0-2.71 | 0.77 ± 0.67 | 0-4 |
| Never smoked | 0.21 | 0-1 | 0.09 | 0-1 |

| | | |
|---|---|---|
| Total missing data, all | 8.81% | 8.73% |
| variables and waves | | |

CPD, cigarettes per day.
Note:
Due to extremely small coefficient sizes, CPD was specified as CPD/20, thus making the measurement equivalent to packs per day; FEV₁, forced expiratory volume in 1 second; SD, standard deviation.
*Descriptive statistics calculated from non-imputed data at participant's first assessment.

In developing the random effect-based outcome measures, this embodiment systematically developed linear mixed models predicting FEV₁. Linear mixed models are a generalization of linear regression allowing for the inclusion of random deviations (i.e. random effects) other than those associated with the overall residual term. In matrix notation,

y=Xβ+Zu+ε

where y is the n×1 vector of responses, X is a n×p design/covariate matrix for the fixed effect β, and Z is the n×q design/covariate matrix for the random effects u. The n×1 vector of residuals E is assumed to be multivariate normal with mean zero and variance matrix σₑ²Iₙ.

The fixed portion, Xβ, is equivalent to the linear predictor of OLS regression. For the random portion, Zu+ε, it is assumed that the u has variance-covariance matrix G and that u is orthogonal to ε so that

$$Var [ \begin{matrix}u \\ ɛ\end{matrix} ] = [ \begin{matrix}G & 0 \\ 0 & {σ}_{e}^{2} {I}_{n}\end{matrix} ]$$

The random effects u are not directly estimated (although, as described below, they may be predicted), but instead are characterized by the elements of G, known as the variance components, that are estimated along with the residual variance σₑ². Considering Zu+ε the combined error, this embodiment shows that y is multivariate normal with mean Xβ and n×n variance-covariance matrix

V=ZGZ′+σₑ²Iₙ

The model building process is shown in Table 6. The outcome measures used in this analysis were derived from the random effects of the final, best-fitting model:

yᵢⱼ=β₀+β₁x₁ᵢⱼ+β₂x₂ᵢⱼ+β₃x₃ᵢⱼ+β₄x₄ᵢⱼ+β₅x₅ᵢⱼ+β₆x₆ᵢⱼ+β₇x₇ᵢⱼ+u₀ᵢ+u₁ᵢ+u₂ᵢ+u₃ᵢ+eᵢⱼ

where i indexes subjects, j indexes repeated assessments, y is FEV₁, β₀ is the intercept fixed effect, x₁ is age, β₁ is the age fixed effect, x₂ is pack-years, β₂ is the pack-years fixed effect, x₃ is CPD x age, β₃ is the CPD x age fixed effect, x₄ is height, β₄ is the height fixed effect, x₅ is gender, β₅ is the gender fixed effect, x₆ is gender x age, β₆ is the gender x age fixed effect, x₇ is never-smoked status, β₇ is the never-smoked status fixed effect, u₀ᵢ is the intercept random effect, u₁ᵢ is the age random effect, u₂ᵢ is the pack-years random effect, u₃ᵢ is the CPD x age random effect and eᵢⱼ is the within-subject residual. Parameter estimates and p-values for the final model are shown in Table 6 as Model 15 and in Table 7 respectively.

TABLE 6
Results of FEV₁ linear mixed modeling

| | | Test | | vs. | |
|---|---|---|---|---|---|
| Model | Variables | statistic* | df^{†} | Model | p-value |
| 1 | Intercept | — | — | — | — |
| 2 | Model 1 + Random Intercept | 2423.13 | 1, 41 | 1 | <.001 |
| 3 | Model 2 + Age | 992.28 | 1, 25 | 2 | <.001 |
| 4 | Model 3 + Random Age | 99.30 | 1, 159 | 3 | <.001 |
| | Model 4 + Unstructured RE | | | | |
| 5 | covariance | 122.74 | 1, 128 | 4 | <.001 |
| 6 | Model 4 + Age² | 2.48 | 1, 17 | 5 | NS |
| 7 | Model 5 + Height | 283.98 | 1, 110 | 5 | <.001 |
| 8 | Model 6 + Male | 26.38 | 1, 137 | 7 | <.001 |
| 9 | Model 7 + Male × Age | 15.00 | 1, 1144 | 8 | <.001 |
| 10 | Model 8 + Height × Age | 3.80 | 1, 65 | 9 | NS |
| 11 | Model 8 + Pack-years | 14.56 | 1, 6 | 9 | <.01 |
| 12 | Model 10 + Random Pack-years | 51.35 | 1, 7 | 11 | <.001 |
| 13 | Model 11 + CPD × Age | 7.89 | 1, 7 | 12 | <.05 |
| 14 | Model 11 + Random CPD × Age | 27.96 | 1, 18 | 13 | <.001 |
| 15 | Model 12 + Never smoked | 104.69 | 1, 248 | 14 | <.001 |
| 16 | Model 13 + CPD | 1.03 | 1, 41 | 15 | NS |
| 17 | Model 13 + Pack-years × Age | 0.46 | 1, 164 | 15 | NS |
| 18 | Model 13 + Never smoked × Age | 0.36 | 1, 19779 | 15 | NS |

CPD, cigarettes per day.
Note:
Due to extremely small coefficient sizes, CPD was specified as CPD/20, thus making the measurement equivalent to packs per day; FEV₁ forced expiratory volume in 1 second; RE, random effect; NS, not significant.
*This is the multiple imputation version of the likelihood ratio test statistic (Allison, P. (2002) Missing data. Sage Publications, Inc., Thousand Oaks, CA; Li, el al., 1991. JASA, 86, 1065-1073). The test statistic approximates an F-distribution under the null hypothesis. See Bollen and Curran (Bollen and Curran (2006) Latent curve models: A structural equation approach. Wiley, Hoboken, NJ) for test statistic and degrees of freedom equations.
^{†}Two values are given for the degrees of freedom as the test statistic has an F-distribution.

The covariance structure of the four random effects was modeled as unstructured:

$$[ \begin{matrix}{u}_{0 i} \\ {u}_{1 i} \\ {u}_{2 i} \\ {u}_{3 i}\end{matrix} ] \sim N ( 0 , G )$$

$$with$$

$$G = [ \begin{matrix}{σ}_{u 0}^{2} & & & \\ {σ}_{u 10} & {σ}_{u 1}^{2} & & \\ {σ}_{u 20} & {σ}_{u 21} & {σ}_{u 2}^{2} & \\ {σ}_{u 30} & {σ}_{u 31} & {σ}_{u 32} & {σ}_{u 3}^{2}\end{matrix} ]$$

Thus, the random parameters are multivariate normal distributed with means of zero and variance-covariance matrix G. The variances of the parameters are on the diagonal and the covariances in the off-diagonal cells of G. The residual is assumed to be normally distributed with a mean of zero and variance of σ²ₑ.

Because random effects are not directly estimated by the mixed model, they must be predicted in an additional post-estimation step. BLUPs of the random effects u were obtained as

ũ=\tilde{G}Z′\tilde{V}⁻¹(y−X\hat{β})

where \tilde{G} and \tilde{V} are G and V with estimates of the variance components plugged in. The EM algorithm was used for maximum likelihood estimation as described by Pinheiro and Bates (Pinheiro and Bates (2000) Mixed-effects models in S and S-plus. Springer, N.Y.).

TABLE 7
Parameter estimates and statistical significance
of final linear mixed model of FEV₁

| | Parameters | SE | p-value |
|---|---|---|---|
| Fixed Effects | | | |
| Intercept (L) | 2.960 | 0.047 | <.001 |
| Age (y) | −0.027 | 0.002 | <.001 |
| Height (cm) | 0.031 | 0.002 | <.001 |
| Male Gender | 0.542 | 0.055 | <.001 |
| Height × Age | −0.009 | 0.002 | <.001 |
| Pack-years | −0.002 | 0.001 | <.05 |
| CPD × Age | −0.003 | 0.000 | <.01 |
| Never smoked | 0.780 | 0.064 | <.001 |
| Random Effects | | | |
| SD (Intercept) | 0.505 | 0.031 | <.001 |
| SD (Age) | 0.021 | 0.001 | <.001 |
| SD (Pack-years) | 0.008 | 0.002 | <.001 |
| SD (CPD × Age) | 0.007 | 0.001 | <.001 |

CPD, cigarettes per day.
Note:
Due to extremely small coefficient sizes, CPD was specified as CPD/20, thus making the measurement equivalent to packs per day; FEV₁, forced expiratory volume in 1 second; SD, standard deviation; SE, standard error.

The claims below are not restricted to the particular embodiments or examples, which are provided for illustrative purposes, and are not intended to limit the methods and compositions of the present disclosure in any manner. Those of skill in the art will recognize a variety of parameters that can be changed or modified to yield the same or similar results.

1. A method for diagnosing or prognosing a lung disease or impaired lung function, or predicting the likelihood of developing a lung disease or impaired lung function, comprising examining the methylation of CpG sites within two or more genes selected from the CCR5 gene and the genes listed in Table 2 or Table 3;
wherein said lung disease is selected from the group consisting of obstructive pulmonary disease, chronic systemic inflammation, emphysema, asthma, pulmonary fibrosis, cystic fibrosis, obstructive lung disease, pulmonary inflammatory disorder, and COPD.

2. The method of claim 1, wherein said one or more genes are 3 or more, 5 or more, 6 or more, 8 or more, 10 or more, 12 or more, 15 or more, 20 or more, 25 or more, or 30 or more genes selected from the genes listed in Table 2, Table 3, and the CCR5 gene.

3. The method of claim 1, wherein said one or more genes are listed in Table 2.

4. (canceled)

5. The method of claim 1, wherein said one or more genes are listed in Table 3.

6. The method of claim 1, wherein said two or more genes are associated with CPD x age-decline.

7. The method of claim 1, wherein said one or more genes include at least one, at least two, at least three, or at least four genes wherein the methylation status of each gene is associated with pack-year decline and age decline.

8. The method of claim 1, wherein said methylation of CpG sites within one or more genes are selected from the gene comprising: CCR5_P630_R, ACVR1C_P363_F; ATP10A_P147_F; HTR1B_P222_F; KIAA1804_P689_R; SOX1_P294_F; and TRIP6_P1274_R.

9. A composition comprising two or more nucleic acid molecules;
each of said two or more nucleic acid molecules comprising a first nucleic acid sequence and an optional second nucleic acid sequence;
wherein said first nucleic acid sequence in each of said two or more nucleic acid molecules comprises a nucleic acid sequence having at least 20 contiguous nucleotides encompassing a CpG site of a different gene listed in Table 2 or Table 3, and wherein a first portion of the first nucleic sequence of at least one of said two or more nucleic acid molecules differs in its methylation of at least one CpG site from a second portion said at least one of said two or more nucleic acid molecules.

10-17. (canceled)

18. The composition claim 9, wherein said composition comprises a spatially addressable array, wherein said spatially addressable array comprises two or more locations each having at least one of said two or more nucleic acid molecules present.

19-21. (canceled)

22. The composition of claim 18, wherein said second nucleic acid sequence comprises a sequence that can hybridize to said location on said array.

23-24. (canceled)

25. A method for diagnosing or prognosing a lung disease or impaired lung function, predicting the likelihood of developing a lung disease or impaired lung function, or of prognosing a decline in lung function as assessed by a decline in the ratio of FEV1 to FVC comprising examining the methylation of one or more CpG of two or more genes selected from the genes listed in Table 2, the genes listed in Table 3, and the CCR5 gene; wherein methylation of said one or more CpG sites each show a statistically significant correlation with said lung disease or impaired lung function and/or said decline in the ration of FEV1 to FVC; and
wherein said lung disease is selected from the group consisting of obstructive pulmonary disease, chronic systemic inflammation, emphysema, asthma, pulmonary fibrosis, cystic fibrosis, obstructive lung disease, pulmonary inflammatory disorder, and COPD.

26-27. (canceled)

28. A method for detecting, predicting or prognosing a lung disease or impaired lung function, comprising:
a) examining the methylation of a nucleic acid sample of a subject at one or more sites in a gene selected from those genes listed in Table 2 or Table 3,
b) comparing a profile of the methylation of said sites in said gene with a profile of methylation of the site in said gene in a standard sample, wherein the comparison identifies the subject as having a disease or a predisposition to a disease or disorder that is associated with a decline in lung function; and
wherein said lung disease is selected from the group consisting of obstructive pulmonary disease, chronic systemic inflammation, emphysema, asthma, pulmonary fibrosis, cystic fibrosis, obstructive lung disease, pulmonary inflammatory disorder, and COPD.

29. A method for detecting the presence or predisposition to developing a disease or disorder associated with a decline in lung function comprising:
a) obtaining a methylation profile of a biological sample of a subject wherein said sample includes at least
one nucleic acid sequence having one or more CpG sites and wherein the methylation profile is defined as a test profile; and
c) comparing the methylation profile of the test sample relative to the methylation profile of a standard sample, wherein the comparison identifies the subject as having a disease or a predisposition to a disease or disorder that is associated with a decline in lung function;
wherein said lung disease is selected from the group consisting of obstructive pulmonary disease, chronic systemic inflammation, emphysema, asthma, pulmonary fibrosis, cystic fibrosis, obstructive lung disease, pulmonary inflammatory disorder, and COPD.

30-41. (canceled)

42. A method for monitoring the course of progression, or managing the treatment of a lung disease in a subject comprising:
a) measuring the methylation of at least one CpG site in a first biological sample from the subject;
b) measuring the methylation of said CpG site in a second biological sample from the subject, wherein the second biological sample is obtained from the subject after the first biological sample; and
c) correlating the measurements with a progression or regression of lung disease in the subject, where an increase in methylation in said CpG site in the second sample relative to said first sample is indicative of disease progression and a reduction in the methylation is indicative of disease regression;
wherein said lung disease is selected from the group consisting of obstructive pulmonary disease, chronic systemic inflammation, emphysema, asthma, pulmonary fibrosis, cystic fibrosis, obstructive lung disease, pulmonary inflammatory disorder, and COPD.

43. The method of claim 42, wherein said CpG site is present in a gene selected from the CCR5 gene and those genes listed in Table 2 and/or Table 3.

44. The method of claim 43, wherein methylation sites within said genes are selected from: CCR5_P630_R, ACVR1C_P363_F; ATP10A_P147_F; HTR1B_P222_F; KIAA1804_P689_R; SOX1_P294_F; and TRIP6_P1274_R.

45. The method of any of claim 42, comprising measuring in said first and/or said second biological sample the methylation of at least two CpG site in at least two different genes selected from the CCR5 gene and those genes listed in Table 2 and/or Table 3.

46. (canceled)

47. The method of claim 42, wherein at least one therapeutic agent was administered to said subject, wherein said at least one therapeutic agent is administered after said first biological sample was obtained from said subject, and before said second biological sample was obtained from said subject.

49-52. (canceled)
